(19) United States
(12) Patent Application Publication
(10) Pub. No.: US 2012/0171229 A1
(43) Pub. Date: Jul. 5, 2012

(54) SYNTHETIC NANOCARRIERS WITH REACTIVE GROUPS THAT RELEASE BIOLOGICALLY ACTIVE AGENTS

(75) Inventors: Charles Zepp, Hardwick, MA (US); Yun Gao, Southborough, MA (US)

(73) Assignee: Selecta Biosciences, Inc., Watertown, MA (US)

(21) Appl. No.: 13/341,020

(22) Filed: Dec. 30, 2011

Related U.S. Application Data
(60) Provisional application No. 61/428,317, filed on Dec. 30, 2010.

(51) Int. Cl. A61K 47/48; C07H 23/00; A61K 39/00; A61P 37/02; A61P 31/12; C08G 63/08; B32B 15/02; C07K 17/02; A61P 35/00; B82Y 5/00; B82Y 30/00

(52) U.S. Cl. 424/184.1; 530/409; 536/23.1; 424/78.17; 514/44 R; 514/1.1; 525/450; 428/402.24; 977/773; 977/906

(57) ABSTRACT

This invention relates to compositions, and related compounds and methods, of conjugates of synthetic nanocarriers, or components thereof, and biologically active agents, such as immunomodulatory agents, antigens, anticancer agents or antiviral agents. The biologically active agents are released from the synthetic nanocarriers in the presence of a reducing agent or by reaction with a thiol.

### RELATED APPLICATIONS

This application claims the benefit under 35 U.S.C. §119 of U.S. provisional application 61/428,317, filed Dec. 30, 2010, the entire contents of which are incorporated herein by reference.

### FIELD OF THE INVENTION

This invention relates to compositions, and related compounds and methods, of conjugates of synthetic nanocarriers, or components thereof, and biologically active agents. The biologically active agents may be released from the synthetic nanocarriers under reducing conditions or by reaction with thiols.

### BACKGROUND OF THE INVENTION

It is at times advantageous to release biologically active agents from delivery vehicles in cells or cellular compartments, such as in the endosome or lysosome. Currently, attachment chemistries often require reactive groups that release biologically active agents in a pH-dependent manner. There is a need, therefore, for new compositions where biologically active agents are conjugated to delivery vehicles in a manner that allows for more universal release, particularly in cells or cellular compartments.

### SUMMARY OF THE INVENTION

In one aspect, a composition, comprising a compound comprising the structure of formula (I) Q-X—Y (I), where Q comprises a synthetic nanocarrier, X comprises a reactive moiety that is reduced in the presence of a reducing agent or reacts with a thiol, resulting in the release of Y from Q, and Y comprises a biologically active agent is provided. In one embodiment, the reactive moiety that is reduced in the presence of a reducing agent or reacts with a thiol comprises a disulfide linkage or quinone. In another embodiment, the reducing agent is NADH, NADPH, or a quinone reductase enzyme.

In another embodiment, the compound comprises the structure of formula (II)

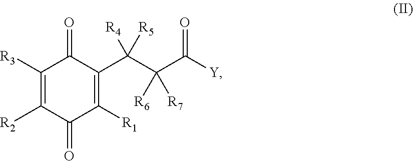

where at least one of R₁-R₇ comprises Q; R₁ comprises Q, H, an unsubstituted or substituted alkyl, an unsubstituted or substituted aryl, alkoxy or halogen; R₂ and R₃ each is H or comprise Q, an unsubstituted or substituted alkyl, an unsubstituted or substituted aryl, alkoxy or halogen or are joined to form a cyclic ring; R₄ and R₅ each comprise Q, an unsubstituted or substituted alkyl, an unsubstituted or substituted aryl, alkoxy or halogen, except that both R₄ and R₅ do not comprise Q; and R₆ and R₇ each is H or comprise Q, an unsubstituted or substituted alkyl, an unsubstituted or substituted aryl, alkoxy or halogen. In another embodiment, R₆ and R₇ each is a methyl group.

In another embodiment, the compound comprises the structure of formula (III)

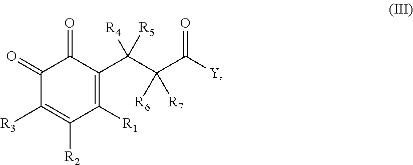

where at least one of R₁-R₇ comprises Q; R₁ comprises Q, H, an unsubstituted or substituted alkyl, an unsubstituted or substituted aryl, alkoxy or halogen; R₂ and R₃ each is H or comprise Q, an unsubstituted or substituted alkyl, an unsubstituted or substituted aryl, alkoxy or halogen or are joined to form a cyclic ring; R₄ and R₅ each comprise Q or an unsubstituted or substituted alkyl, an unsubstituted or substituted aryl, alkoxy or halogen, except that both R₄ and R₅ do not comprise Q; and R₆ and R₇ each is H or comprise Q, an unsubstituted or substituted alkyl, an unsubstituted or substituted aryl, alkoxy or halogen. In another embodiment, R₄ and R₅ each is a methyl group.

In another embodiment, the reactive moiety comprises a disulfide linkage coupled to a self-immolating group.

In another embodiment, Q comprises a lipid-based nanoparticle, polymeric nanoparticle, metallic nanoparticle, dendrimer, buckyball, nanowire, peptide or protein-based nanoparticle, virus-like particle or lipid-polymer nanoparticle. In another embodiment, Q comprises a polymeric nanoparticle. In another embodiment, Q comprises a metallic nanoparticle. In another embodiment, the metallic nanoparticle is a gold nanoparticle.

In another embodiment, Y comprises an immunomodulating agent, an anticancer agent or an antiviral agent. In another embodiment, the immunomodulating agent is a TLR agonist or CpG-containing oligonucleotide. In another embodiment, the TLR agonist comprises an imidazoquinoline or adenine compound. In another embodiment, the imidazoquinoline is imiquimod. In another embodiment, the imidazoquinoline is resiquimod. In another embodiment, the adenine compound is an 8-oxoadenine.

In another embodiment, Y is encapsulated within the synthetic nanocarrier. In another embodiment, Y is on the surface of the synthetic nanocarrier. In another embodiment, Y is encapsulated within and on the surface of the synthetic nanocarrier.

In another aspect, any of the compounds provided can be comprise in a composition. Such compositions are also provided.

In another embodiment, the compositions provided further comprise a pharmaceutically acceptable excipient.

In another embodiment, the compositions provided further comprise another biologically active agent. In another embodiment, the other biologically active agent is at least one antigen.

In another aspect, any of the compounds or compositions provided can be comprised in a vaccine. Such vaccines are also provided.

In another aspect, any of the compounds or compositions provided can be comprised in a dosage form. Such dosage forms are also provided.

In another aspect, a method comprising administering any of the compounds, compositions, vaccines or dosage forms to a subject if provided. In another embodiment, the method is for modulating an immune response. In another embodiment, the method is for inducing or enhancing an immune response. In another embodiment, the method further comprises administering another biologically active agent to the subject.

In another aspect, any of the compounds, compositions, vaccines or dosage forms may be for use in therapy or prophylaxis.

In another aspect, any of the compounds, compositions, vaccines or dosage forms may be for use in any of the methods provided.

In another aspect, any of the compounds, compositions, vaccines or dosage forms may be for use in a method of modulating, for example inducing, enhancing, suppressing, directing, or redirecting, an immune response.

In another aspect, any of the compounds, compositions, vaccines or dosage forms may be for use in a method of diagnosis, prophylaxis and/or treatment of cancer, infectious disease, metabolic disease, degenerative disease, autoimmune disease, inflammatory disease, immunological disease, an addiction, or a condition resulting from the exposure to a toxin, hazardous substance, environmental toxin, or other harmful agent.

In another aspect, any of the compounds, compositions, vaccines or dosage forms may be for use in a method of therapy or prophylaxis comprising administration by the subcutaneous, intramuscular, intradermal, oral, intranasal, transmucosal, sublingual, rectal, ophthalmic, transdermal, transcutaneous routes, or by a combination of these routes.

In another aspect, use of any of the compounds, compositions, vaccines or dosage forms for the manufacture of a medicament for use in any of the methods provided is provided.

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “the synthetic nanocarrier” includes a mixture of two or more such synthetic nanocarriers or a plurality of such synthetic nanocarriers, reference to “a DNA molecule” includes a mixture of two or more such DNA molecules or a plurality of such DNA molecules, reference to “an adjuvant” includes a mixture of two or more such materials or a plurality of adjuvant molecules, and the like.

### A. INTRODUCTION

The inventors have unexpectedly and surprisingly discovered that the problems and limitations noted above can be overcome by practicing the invention disclosed herein. In particular, the inventors have unexpectedly discovered that it is possible to provide compositions, and related methods, that comprise a synthetic nanocarrier coupled to a biologically active agent via a reactive moiety that is reduced in the presence of a reducing agent or that reacts with a thiol, resulting in the release of the biologically active agent from the synthetic nanocarrier. The reactive moieties that are reduced in the presence of a reducing agent or reacts with a thiol include moieties that comprise a disulfide linkage or quinone. Compositions that include such reactive groups offer more universal release of compounds, such as biologically active agents, and release under this mechanism is particularly efficient within the cell and cellular compartments, such as the endosome or lysosome.

The compositions provided herein, therefore, are attractive for the delivery of biologically active agents. The property of being reduced or reactive with the aforementioned agents (i.e., reducing agents or thiols) targets the biologically active agents to cells, and in particular to the endosomal or lysosomal compartment of cells. Release in the target compartment allows the biologically active agents to be free to act intracellularly (e.g., with receptors) and effect a desired biological response (e.g., an immune response). As a result, the compositions provided herein can effectively result in a biological response while reducing off-target effects (e.g., adverse events) and/or result in a biological response at lower concentrations.

Accordingly, in one aspect, a composition is provided that comprises a compound comprising the structure of formula (I), Q-X—Y (I), where Q comprises a synthetic nanocarrier, X comprises a reactive moiety that is reduced in the presence of a reducing agent or reacts with a thiol, resulting in the release of Y from Q, and Y comprises a biologically active agent.

Reactive moieties that are reduced in the presence of a reducing agent and result in the release of a biologically active agent from a synthetic nanocarrier include moieties that comprise a disulfide linkage or a quinone. Reducing agents, as used herein, are hydride donors, such as NADH, NADPH, and quinone reductase enzyme.

Accordingly, in some embodiments, the reactive moiety comprises a disulfide. In other embodiments, the reactive moiety further comprises a self-immolating group. Such reactive moieties can be directly coupled to the synthetic nanocarrier and/or the biologically active agent or they can be indirectly coupled to the synthetic nanocarrier and/or the biologically active agent by the use of a linker that is coupled to the synthetic nanocarrier and/or the biologically active agent.

In other embodiments, the quinone is a para-benzoquinone or 1,4-benzoquinone, and the compound that comprises the structure of formula (I) has the following structure (formula (II)):

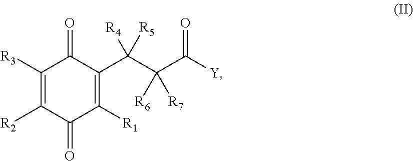

where at least one of R₁-R₇ comprises Q; R₁ comprises Q, H, an unsubstituted or substituted alkyl, an unsubstituted or substituted aryl, alkoxy or halogen; R₂ and R₃ each is H or comprise Q, an unsubstituted or substituted alkyl, an unsubstituted or substituted aryl, alkoxy or halogen or are joined to form a cyclic ring; R₄ and R₅ each comprise Q, an unsubstituted or substituted alkyl, an unsubstituted or substituted aryl, alkoxy or halogen, except that both R₄ and R₅ do not comprise Q; and R₆ and R₇ each is H or comprise Q, an unsubstituted or substituted alkyl, an unsubstituted or substituted aryl, alkoxy or halogen.

In still other embodiments, the quinone is an ortho-benzoqinone, such as 1,2-benzoquinone, and the compound that comprises the structure of formula (I) has the following structure (formula (III)):

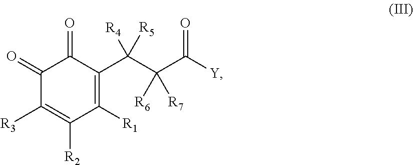

where at least one of R₁-R₇ comprises Q; R₁ comprises Q, H, an unsubstituted or substituted alkyl, an unsubstituted or substituted aryl, alkoxy or halogen; R₂ and R₃ each is H or comprise Q, an unsubstituted or substituted alkyl, an unsubstituted or substituted aryl, alkoxy or halogen or are joined to form a cyclic ring; R₄ and R₅ each comprise Q or an unsubstituted or substituted alkyl, an unsubstituted or substituted aryl, alkoxy or halogen, except that both R₄ and R₅ do not comprise Q; and R₆ and R₇ each is H or comprise Q, an unsubstituted or substituted alkyl, an unsubstituted or substituted aryl, alkoxy or halogen.

Reactive moieties that react with a thiol and result in the release of a biologically active agent from a synthetic nanocarrier include moieties that comprise a quinone. Thiols include compounds, such as cysteine or glutathione.

Accordingly, in some embodiments, the quinone is a para-benzoquinone, 1,4-benzoquinone or ortho-benzoquinone as provided above, and the compound that comprises the structure of formula (I) has the structure of formula (II) or formula (III), respectively. For thiol addition reactions with these quinones, at least one of the R groups of the quinone is H, while for thiol reduction reactions, the quinone can be fully substituted

The invention will now be described in more detail below.

### B. DEFINITIONS

“Administering” or “administration” means providing a drug to a subject in a manner that is pharmacologically useful.

“Anticancer agent” means any therapeutic agent that results in a biological response that is beneficial in the treatment of cancer. Such a biological response includes a reduction in tumor size, a reduction in the number and/or size of metastases, a reduction in the number of cancer cells, an alleviation or elimination of one or more symptoms of a subject with cancer, etc. Anticancer agents include cytotoxic radionuclides, chemical toxins, protein toxins and agents that act on tumor vasculature. Chemical toxins include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other anticancer agents include dolastatins (U.S. Pat. Nos. 6,034,065 and 6,239,104) and derivatives thereof. Other anticancer agents include poisonous lectins, plant toxins such as ricin, abrin, modeccin, botulina and diphtheria toxins. Agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein) and interferon inducible protein 10 (U.S. Pat. No. 5,994,292). A number of antiangiogenic agents currently in clinical trials are also contemplated. Additional antiangiogenic agents are described by Kerbel, J. Clin. Oncol. 19(18s):45s-51s, 2001, which is incorporated by reference herein. Further anticancer agents are known to those skilled in the art.

“Antigen” means a B cell antigen or T cell antigen. In embodiments, antigens are coupled to the synthetic nanocarriers. In other embodiments, antigens are not coupled to the synthetic nanocarriers. In embodiments, antigens are coadministered with the synthetic nanocarriers. In other embodiments, antigens are not coadministered with the synthetic nanocarriers. “Type(s) of antigens” means molecules that share the same, or substantially the same, antigenic characteristics.

“Antiviral agent” means any therapeutic agent that results in a biological response that is beneficial in the treatment or prevention of a viral infection. Such a biological response includes an immune response against a virus or a protein associated therewith, a reduction in the viral load in a subject, a reduction in the infectivity of a virus in a subject, an alleviation or elimination of one or more symptoms of a subject with a viral infection, etc. Antiviral agents include nucleotide analogues which include acyclovir, gancyclovir, idoxuridine, ribavirin, dideoxyinosine, dideoxycytidine and zidovudine (azidothymidine). Further antiviral agents are known to those skilled in the art.

“At least a portion of the dose” means at least some part of the dose, ranging up to including all of the dose.

“Biologically active agent” means any agent that results in some biological response, such as a pharmaceutical, therapeutic and/or immune response. Biologically active agents include therapeutic drugs, antigens as well as immunomodulatory agents. Therapeutic drugs include anticancer agents and antiviral agents. The biologically active agent of the inventive synthetic nanocarrier conjugates are coupled to the synthetic nanocarriers via a reactive moiety that is reduced in the presence of a reducing agent or that reacts with a thiol, resulting in the release of the biologically active agent from the synthetic nanocarrier. Additional biologically active agents can also be included in the synthetic nanocarriers and compositions provided herein. Such other biologically active agents can be coupled to synthetic nanocarriers by any method provided herein or otherwise known to those of ordinary skill in the art. Such other biologically active agents, in some embodiments, are not coupled to the inventive synthetic nanocarrier conjugates.

“Couple” or “Coupled” or “Couples” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of coupling.

“Dosage form” means a pharmacologically and/or immunologically active material in a medium, carrier, vehicle, or device suitable for administration to a subject.

“Encapsulate” means to enclose within a synthetic nanocarrier, preferably enclose completely within a synthetic nanocarrier. Most or all of a substance that is encapsulated is not exposed to the local environment external to the synthetic nanocarrier. Encapsulation is distinct from absorption, which places most or all of a substance on a surface of a synthetic nanocarrier, and leaves the substance exposed to the local environment external to the synthetic nanocarrier. The biologically active agents of the inventive synthetic nanocarrier conjugates can be encapsulated within and/or present on the surface of the synthetic nanocarriers. In some embodiments, such as with metallic synthetic nanocarriers, the biologically active agents are only on the surface of the synthetic nanocarrier. In other embodiments, such as with polymeric synthetic nanocarriers, the biologically active agents are both encapsulated and on the surface of the synthetic nanocarrier. The inventive synthetic nanocarrier conjugates can also comprise additional biologically active agents, which can be encapsulated within and/or present on the surface of the synthetic nanocarriers.

“Hydride donor”, as used herein, is a compound with one or more hydrogen centers capable of donating a hydride with reducing properties. Such hydride donors include but are not limited to NADH and NADPH, or a quinone reductase enzyme.

“Immunomodulatory agent” means an agent that modulates an immune response. Such an agent does not constitute a specific antigen, but can modulate an immune response to an antigen, such as a concomitantly administered antigen when also administered in some embodiments. “Modulate”, as used herein, refers to inducing, enhancing, stimulating, directing or redirecting, etc. an immune response. Such agents include immunostimulatory agents that stimulate (or boost) an immune response to an antigen but is not an antigen or derived from an antigen. In some embodiments, the immunomodulatory agent is on the surface of the synthetic nanocarrier and/or is encapsulated within the synthetic nanocarrier. Generally, the immunomodulatory agent is coupled to the synthetic nanocarrier via the reactive moieties provided.

In some embodiments, all of the immunomodulatory agents of a synthetic nanocarrier are identical to one another. In some embodiments, a synthetic nanocarrier comprises a number of different types of immunomodulatory agents. In some embodiments, a synthetic nanocarrier comprises multiple individual immunomodulatory agents, all of which are identical to one another. In some embodiments, a synthetic nanocarrier comprises exactly one type of immunomodulatory agent. In some embodiments, a synthetic nanocarrier comprises exactly two distinct types of immunomodulatory agents. In some embodiments, a synthetic nanocarrier comprises greater than two distinct types of immunomodulatory agents.

Immunomodulatory agents include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIX™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments.

In embodiments, immunomodulatory agents comprise agonists for pattern recognition receptors (PRR), including, but not limited to Toll-Like Receptors (TLRs), specifically TLRs 2, 3, 4, 5, 7, 8, 9 and/or combinations thereof. In other embodiments, immunomodulatory agents comprise agonists for Toll-Like Receptors 3, agonists for Toll-Like Receptors 7 and 8, or agonists for Toll-Like Receptor 9; preferably the recited immunomodulatory agents comprise imidazoquinolines; such as R848; adenine derivatives, such as those disclosed in U.S. Pat. No. 6,329,381 (Sumitomo Pharmaceutical Company), US Published Patent Application 2010/0075995 to Biggadike et al., WO 2010/018134, WO 2010/018133, WO 2010/018132, WO 2010/018131, WO 2010/018130 and WO 2008/101867 to Campos et al.; immunostimulatory DNA; or immunostimulatory RNA. In specific embodiments, synthetic nanocarriers incorporate as immunomodulatory agents that are agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred immunomodulatory agents comprise imiquimod and resiquimod (also known as R848). In specific embodiments, an immunomodulatory agent may be an agonist for the DC surface molecule CD40. In certain embodiments, to stimulate immunity rather than tolerance, a synthetic nanocarrier incorporates an immunomodulatory agent that promotes DC maturation (needed for priming of naive T cells) and the production of cytokines, such as type I interferons, which promote antibody immune responses. In embodiments, immunomodulatory agents also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an immunomodulatory agent may be a TLR-4 agonist, such as bacterial lipopolysacccharide (LPS), VSV-G, and/or HMGB-1. In some embodiments, immunomodulatory agents may comprise TLR-5 agonists, such as flagellin, or portions or derivatives thereof, including but not limited to those disclosed in U.S. Pat. Nos. 6,130,082, 6,585,980, and 7,192,725. In specific embodiments, synthetic nanocarriers incorporate a ligand for Toll-like receptor (TLR)-9, such as immunostimulatory DNA molecules comprising CpGs, which induce type I interferon secretion, and stimulate T and B cell activation leading to increased antibody production and cytotoxic T cell responses (Krieg et al., CpG motifs in bacterial DNA trigger direct B cell activation. Nature. 1995. 374:546-549; Chu et al. CpG oligodeoxynucleotides act as adjuvants that switch on T helper 1 (Th1) immunity. J. Exp. Med. 1997. 186:1623-1631; Lipford et al. CpG-containing synthetic oligonucleotides promote B and cytotoxic T cell responses to protein antigen: a new class of vaccine adjuvants. Eur. J. Immunol. 1997. 27:2340-2344; Roman et al. Immunostimulatory DNA sequences function as T helper-1-promoting adjuvants. Nat. Med. 1997. 3:849-854; Davis et al. CpG DNA is a potent enhancer of specific immunity in mice immunized with recombinant hepatitis B surface antigen. J. Immunol. 1998. 160:870-876; Lipford et al., Bacterial DNA as immune cell activator. Trends Microbiol. 1998. 6:496-500; U.S. Pat. No. 6,207,646 to Krieg et al.; U.S. Pat. No. 7,223,398 to Tuck et al.; U.S. Pat. No. 7,250,403 to Van Nest et al.; or U.S. Pat. No. 7,566,703 to Krieg et al.

In some embodiments, immunomodulatory agents may be proinflammatory stimuli released from necrotic cells (e.g., urate crystals). In some embodiments, immunomodulatory agents may be activated components of the complement cascade (e.g., CD21, CD35, etc.). In some embodiments, immunomodulatory agents may be activated components of immune complexes. The immunomodulatory agents also include complement receptor agonists, such as a molecule that binds to CD21 or CD35. In some embodiments, the complement receptor agonist induces endogenous complement opsonization of the synthetic nanocarrier. In some embodiments, immunomodulatory agents are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

In embodiments, at least a portion of the dose of immunomodulatory agent may be coupled to synthetic nanocarriers, preferably, all of the dose of immunomodulatory agent is coupled to synthetic nanocarriers. In other embodiments, at least a portion of the dose of the immunomodulatory agent is not coupled to the synthetic nanocarriers. In embodiments, the dose of immunomodulatory agent comprises two or more types of immunomodulatory agents. For instance, and without limitation, immunomodulatory agents that act on different TLR receptors may be combined. As an example, in an embodiment a TLR 7/8 agonist may be combined with a TLR 9 agonist. In another embodiment, a TLR 7/8 agonist may be combined with a TLR 9 agonist. In yet another embodiment, a TLR 9 agonist may be combined with a TLR 9 agonist.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of inventive synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 1000:1, still preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a maximum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier sizes is obtained by suspending the synthetic nanocarriers in a liquid (usually aqueous) media and using dynamic light scattering (e.g. using a Brookhaven ZetaPALS instrument).

“Pharmaceutically acceptable excipient” means a pharmacologically inactive material used together with the recited synthetic nanocarriers to formulate the inventive compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Reactive moiety that is reduced by a reducing agent” means a moiety that is reduced by reaction with a hydride donor resulting in the release of a biologically active agent from a synthetic nanocarrier or a component of a synthetic nanocarrier, such as a polymer. Preferably, the reactive moiety comprises a disulfide linkage or quinone as provided herein. Examples of moieties comprising disulfide linkages and/or methods for their preparation are provided elsewhere herein and are described in Vrudhula, et al., Bioorganic and Medicinal Chemistry, 2002, 12: 3591-3594; Chen, et al., Bioconjugate Chem. 2010, 21: 979-987, Zalipsky, et al. Bioconjugate Chem. 2007, 18: 1869-1878; Saito et al., Advanced Drug Delivery Reviews, 2003, 55: 199-215; U.S. Pat. No. 7,238,368; U.S. Pat. No. 6,605,299; U.S. Pat. No. 7,390,780; U.S. Pat. No. 6,849,270; U.S. Pat. No. 7,282,590; WO2005079398; Qiu et al., Chem. Mater. 2004, 16: 850-856; and Carrot et al., Macromolecules 1999, 32: 5264-5269. Examples of quinones and/or methods for their preparation are also provided elsewhere herein and are described in JACS, 94(26), 9175, 1972; JACS, 105(9), 2752, 1983; Pharmaceutical Research, 8(3), 323, 1991; Bioorganic and Medicinal Chemistry Letters, 6(14), 1653, 1996; J. Org. Chem., 62, 1363, 1997; J. Med. Chem., 43, 475, 2000; JACS, 130, 14739, 2008; Biochemistry, 2(3), 537, 1963 and FEBS Letters, 201(2), 296, 1986.

In some embodiments, the reactive moiety is a linker that comprises the disulfide linkage or quinone. In other embodiments, the reactive moiety is a linker that further comprises a self-immolating group in addition to a disulfide linkage. In some embodiments, the reactive moiety can first be coupled to a synthetic nanocarrier before being coupled to the biologically active agent. In other embodiments, the reactive moiety is first coupled to the biologically active agent before being coupled to the synthetic nanocarrier. The reactive moiety can be coupled to the synthetic nanocarrier and/or biologically active agent directly or by coupling to another moiety, including a linker, that is already coupled to the synthetic nanocarrier and/or biologically active agent. Methods of attaching the reactive moieties provided herein to the synthetic nanocarrier, or component thereof, and/or the biologically active agent are provided herein or are otherwise known to those of ordinary skill in the art.

“Reactive moiety that reacts with a thiol” means a moiety that reacts with a thiol resulting in the release of a biologically active agent from a synthetic nanocarrier or a component of a synthetic nanocarrier, such as a polymer. Preferably, the reactive moiety comprises a quinone as provided herein. Examples of quinones and/or methods for their preparation are also provided elsewhere herein and are described in JACS, 94(26), 9175, 1972; JACS, 105(9), 2752, 1983; Pharmaceutical Research, 8(3), 323, 1991; Bioorganic and Medicinal Chemistry Letters, 6(14), 1653, 1996; J. Org. Chem., 62, 1363, 1997; J. Med. Chem., 43, 475, 2000; JACS, 130, 14739, 2008; Biochemistry, 2(3), 537, 1963 and FEBS Letters, 201(2), 296, 1986.

In some embodiments, the reactive moiety is a linker that comprises the quinone. In some embodiments, the reactive moiety can first be coupled to a synthetic nanocarrier before being coupled to the biologically active agent. In other embodiments, the reactive moiety is first coupled to the biologically active agent before being coupled to the synthetic nanocarrier. The reactive moiety can be coupled to the synthetic nanocarrier and/or biologically active agent directly or by coupling to another moiety, including a linker, that is already coupled to the synthetic nanocarrier and/or biologically active agent. Methods of attaching the reactive moieties provided herein to the synthetic nanocarrier, or component thereof, and/or the biologically active agent are provided herein or are otherwise known to those of ordinary skill in the art.

“Reducing agent” means a hydride donor that reacts with a reactive moiety, as provided herein, the result of which reaction releases a biologically active agent from a synthetic nanocarrier.

“Release” or “Release Rate” means the rate that a biologically active agent coupled to a synthetic nanocarrier as provided herein transfers from the synthetic nanocarrier into the local environment, such as a surrounding release media. In embodiments, the local environment, such as the surrounding release media comprises a hydride donor or a thiol, as provided herein. First, the synthetic nanocarrier is prepared for the release testing by placing into the appropriate release media. This is generally done by exchanging a buffer after centrifugation to pellet the synthetic nanocarrier and reconstitution of the synthetic nanocarriers under a mild condition. The assay is started by placing the sample at 37° C. in an appropriate temperature-controlled apparatus. A sample is removed at various time points.

The synthetic nanocarriers are separated from the release media by centrifugation to pellet the synthetic nanocarriers. The release media is assayed for the biologically active agent that has been released from the synthetic nanocarriers. The substance is measured using HPLC to determine the content and quality of the substance. The pellet containing the remaining coupled biologically active agent is dissolved in solvents or hydrolyzed by base to free the coupled biologically active agent from the synthetic nanocarriers. The pellet-contained biologically active agent is then also measured by HPLC after dissolution or destruction of the pellet to determine the content and quality of the biologically active agent that has not been released at a given time point.

The mass balance is closed between biologically active agent that has been released into the release media and what remains coupled to the synthetic nanocarriers. Data are presented as the fraction released or as the net release presented as micrograms released over time.

“Self-immolating group” means a moiety that is cleaved in vivo and when coupled to the biologically active agent transforms it to an inactive state but when cleaved from the biologically active agent transforms it to an active state. In some embodiments, the self-immolating group is coupled directly to the biologically active agent (e.g., via an amide or ester bond). Such groups are described elsewhere herein and are known to those of ordinary skill in the art (See, e.g., El Alaoui, et. al., Bioorganic & Medicinal Chemistry, 14 (2006), 5012-5019; Niculescu-Duvaz, et. al., Methods in Molecular Medicine, Vol. 90, Suicide Gene Therapy: Methods and Reviews, 161-202; Sohn, et al., Polymer Chemistry, 2010, 1, 778-792).

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, inventive synthetic nanocarriers do not comprise chitosan.

A synthetic nanocarrier can be, but is not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid coupled virus-like particles disclosed in published US Patent Application 20060251677 to Bachmann et al., (9) the virus-like particles disclosed in WO2010047839A1 or WO2009106999A2, or (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010). In embodiments, synthetic nanocarriers may possess an aspect ratio greater than 1:1, 1:1.2, 1:1.5, 1:2, 1:3, 1:5, 1:7, or greater than 1:10.

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, when synthetic nanocarriers comprise virus-like particles, the virus-like particles comprise non-natural adjuvant (meaning that the VLPs comprise an adjuvant other than naturally occurring RNA generated during the production of the VLPs).

“Thiol”, also referred to as a mercaptan, as used herein, is an organosulfur compound that contains a carbon-bonded sulfhydryl. Preferably, the thiol has the formula R—SH where R is a substituted or unsubstituted alkyl or substituted or unsubstituted aryl. Thiols include cysteine and glutathione.

“Vaccine” means a composition of matter that improves the immune response to a particular pathogen or disease. A vaccine typically contains factors that stimulate a subject's immune system to recognize a specific antigen as foreign and eliminate it from the subject's body. A vaccine also establishes an immunologic ‘memory’ so the antigen will be quickly recognized and responded to if a person is re-challenged. Vaccines can be prophylactic (for example to prevent future infection by any pathogen), or therapeutic (for example a vaccine against a tumor specific antigen for the treatment of cancer). In embodiments, a vaccine may comprise dosage forms according to the invention.

### C. INVENTIVE COMPOSITIONS

The inventive compositions provided herein are synthetic nanocarriers coupled to biologically active agents via a reactive moiety that is reduced in the presence of a reducing agent or that reacts with a thiol, resulting in the release of the biologically active agents from the synthetic nanocarriers. As mentioned above, such compositions are useful for targeting the biologically active agents to cells and cellular compartments and releasing them therein.

One way of allowing for the release of a biologically active agent is by coupling the biologically active agent to a synthetic nanocarrier with a reactive moiety that comprises a quinone. The biologically active agent will be released when the quinone is reduced to a hydroquinone by, for example, NADH ring closure through the “trialkyl lock” mechanism. Another way of releasing biologically active agents is to convert a quinone amide or ester that is attached to the synthetic nanocarrier to a hydroquinone by reaction with a thiol to initiate ring closure by the trialkyl lock mechanism. Thiols also reduce disulfide linkages.

Orthoquinones also work by the mechanisms described above. In addition to oxygen as in the above moieties, the internal nucleophiles can also be sulfur and nitrogen which will initiate cyclization to form thiolactones and lactams, respectively. These mechanisms are readily adapted to thiol-induced cyclization. When the internal nucleophile is sulfur, the sulfur can be blocked as a disulfide and thereby prevented from initiating cyclization. Reaction with a thiol releases the internal sulfur nucleophile which the initiates cyclization.

Compounds that cyclize by the above mechanisms are described elsewhere herein and/or are known to those of ordinary skill in the art. The uses of these mechanisms to release biologically active agents, such as immunomodulatory agents, from synthetic nanocarriers, however, have not been previously described.

The reactive moieties that are reduced in the presence of a reducing agent include those that comprise a disulfide linkage or quinone as provided herein. The reactive moieties that react with thiols include those that comprise quinones as provided herein. The reactive moieties can be coupled to the synthetic nanocarrier and/or biologically active agent directly or by coupling to a linker or other moiety that is already attached to the synthetic nanocarrier and/or biologically active agent. Examples of such linkers are provided below. In embodiments, the reactive moiety is coupled to a component of the synthetic nanocarrier. For example, when the synthetic nanocarrier is a polymeric nanocarrier, the reactive moiety can be coupled to a polymer of the polymeric nanocarrier.

A non-limiting example of a release reaction of a biologically active agent from a polymer of a polymeric nanocarrier is shown below, where A1 is the biologically active agent. In this example, while it is not required, the reactive moiety comprises a disulfide linkage as well as a self-immolating group (depicted as X). The polymer-biologically active agent conjugate is reacted with glutathione or other thiol containing biomolecules in vivo resulting in the release of the biologically active agent from the polymer. Polymers that form polymeric nanocarriers are provided in more detail below.

Polymer-S-S-X-A1+Glutathione-SH→Polymer-S-S-glutathione+(SX)+H-A1

Other non-limiting examples of disulfide linked polymer-biologically active agent conjugates that react with glutathione or other thiol-containing biomolecules are provided below.

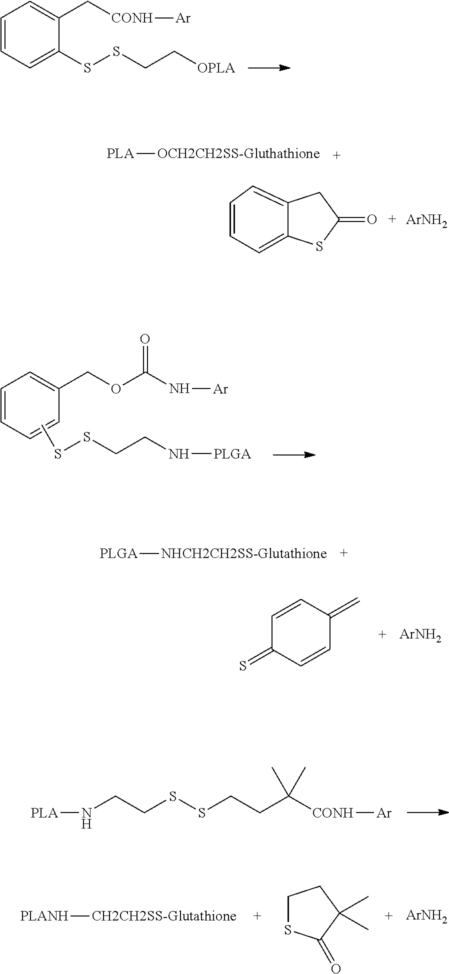

The above polymer-biologically active conjugates can be formed as follows.

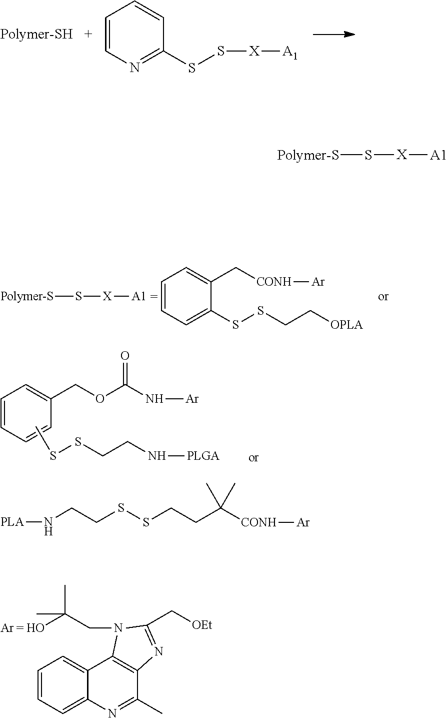

A non-limiting example of the release reaction of a biologically active agent of a compound comprising the structure of formula (II) by a thiol is provided below (where R₁₀ is a substituted or unsubstituted alkyl or substituted or unsubstituted aryl and R₂ is H; the definitions of the other R groups are as provided elsewhere herein).

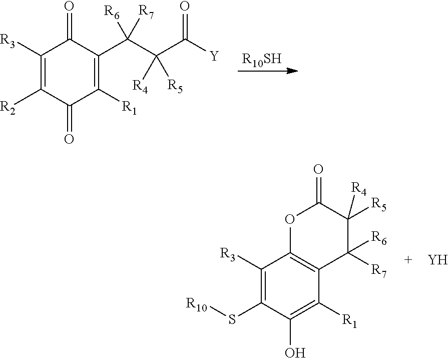

A non-limiting example of the release of a biologically active agent of a compound comprising the structure of formula (II) by a hydride donor is provided below (the definitions of the other R groups are as provided elsewhere herein).

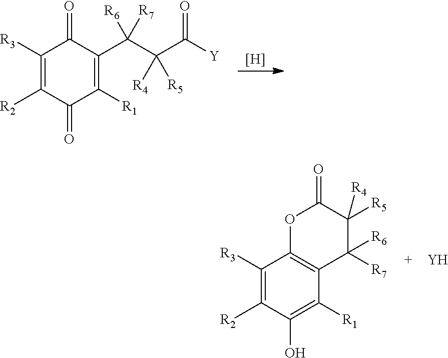

Generally, hydrides react with quinones of formula (II) in the manner outlined below. Again, the particular quinone shown is a non-limiting example of a quinone of formula (II) (again, the R groups are as defined elsewhere herein).

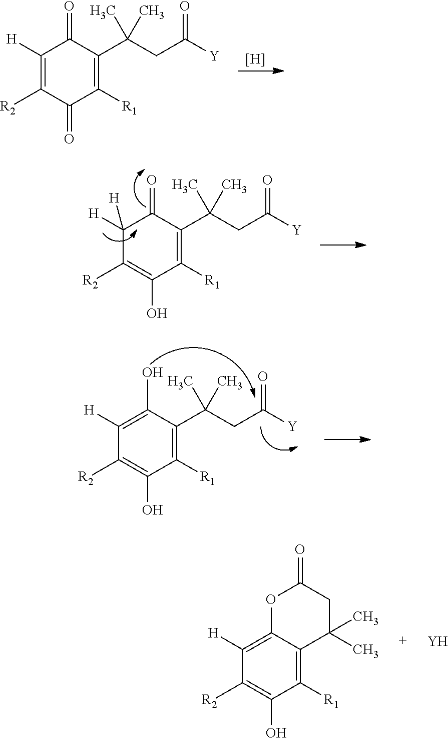

The biologically active agents are coupled to synthetic nanocarriers via any of the reactive moieties provided (directly or indirectly), resulting in the release of the biologically active agents from the synthetic nanocarriers in the presence of a reducing agent or thiol. These synthetic nanocarriers can further include another biologically active agent which likewise can be coupled via the reactive moieties provided or in any other manner known to those of ordinary skill in the art. In some embodiments, the other biologically active agents are not coupled to the inventive synthetic nanocarrier conjugates.

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size, shape, and/or composition so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers. In some embodiments, a population of synthetic nanocarriers may be heterogeneous with respect to size, shape, and/or composition.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In some embodiments, synthetic nanocarriers can comprise one or more polymers. In some embodiments, such a polymer can be surrounded by a coating layer (e.g., liposome, lipid monolayer, micelle, etc.). In some embodiments, various elements of the synthetic nanocarriers can be coupled with the polymer.

In some embodiments, a reactive moiety or biologically active agent provided herein can be covalently associated with a polymeric matrix. In some embodiments, covalent association is mediated by a linker. In some embodiments, an agent can be noncovalently associated with a polymeric matrix. For example, in some embodiments, an agent can be encapsulated within, surrounded by, and/or dispersed throughout a polymeric matrix. Alternatively or additionally, an agent can be associated with a polymeric matrix by hydrophobic interactions, charge interactions, van der Waals forces, etc.

A wide variety of polymers and methods for forming polymeric matrices therefrom are known conventionally. In general, a polymeric matrix comprises one or more polymers. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers suitable for use in the present invention include, but are not limited to polyethylenes, polycarbonates (e.g. poly(1,3-dioxan-2one), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

Polymers also include polypropylene glycol; polythioethers, such as polyethylenesulfides and polypropylenesulfides); polyamines, such as polyethyleneimine; and polyamides, such as poly(2-ethyloxazoline) and polyaminoacids.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g., coupled) to the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In embodiments, the inventive synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)-400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the inventive synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

Compositions according to the invention comprise inventive synthetic nanocarriers in combination with pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, inventive synthetic nanocarriers are suspended in sterile saline solution for injection together with a preservative.

In embodiments, when preparing synthetic nanocarriers as carriers for biologically active agents for use in vaccines, methods for coupling the biologically active agents to the synthetic nanocarriers may be useful. It will be understood to one of ordinary skill in the art that in the coupling methods described below, at least one of the biologically active agents of the synthetic nanocarriers are coupled via a reactive moiety as provided herein (directly or indirectly). It will also be understood to one of ordinary skill in the art that some of the conjugation methods provided herein can be modified to include coupling via the reactive moieties provided. Other biologically active agents coupled to the synthetic nanocarriers are not necessarily coupled via such a reactive moiety and can be coupled by other methods. Such methods are provided herein or are otherwise known to those of ordinary skill.

If the biologically active agent is a small molecule it may be of advantage to attach the biologically active agent to a polymer via the reactive moiety (directly or indirectly) prior to the assembly of the synthetic nanocarriers. In embodiments, it may also be an advantage to prepare the synthetic nanocarriers with surface groups that are used to couple the biologically active agent via the reactive moiety to the synthetic nanocarrier through the use of these surface groups rather than attaching the biologically active agent via the reactive moiety to a polymer and then using this polymer conjugate in the construction of synthetic nanocarriers.

In certain embodiments, the coupling moiety for coupling the biologically active agent via the reactive moiety can be any of the linkers as provided herein or otherwise known in the art that comprise a reactive moiety. In other embodiment, the coupling moiety for coupling another biologically active agent or other element (either via the reactive moiety or not) can be any of the linkers as provided herein or otherwise known in the art (either comprising a reactive moiety or not).

In certain embodiments, the linker is a covalent linker. In embodiments, peptides according to the invention can be covalently coupled to the external surface via a 1,2,3-triazole linker formed by the 1,3-dipolar cycloaddition reaction of azido groups on the surface of the nanocarrier with agent containing an alkyne group or by the 1,3-dipolar cycloaddition reaction of alkynes on the surface of the nanocarrier with agents containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, the covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker and a sulfonamide linker.

An amide linker is formed via an amide bond between an amine on one component such as the agent with the carboxylic acid group of a second component such as the nanocarrier. The amide bond in the linker can be made using any of the conventional amide bond forming reactions with suitably protected amino acids or agents and activated carboxylic acid such N-hydroxysuccinimide-activated ester.

A disulfide linker is made via the formation of a disulfide (S—S) bond between two sulfur atoms of the form, for instance, of R₁—S—S—R₂. A disulfide bond can be formed by thiol exchange of a biologically active agent containing thiol/mercaptan group (—SH) with another activated thiol group on a polymer or nanocarrier or a nanocarrier containing thiol/mercaptan groups with a biologically active agent containing activated thiol group.

A triazole linker, specifically a 1,2,3-triazole of the form

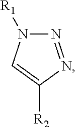

wherein R₁ and R₂ may be any chemical entities, is made by the 1,3-dipolar cycloaddition reaction of an azide attached to a first component such as the nanocarrier with a terminal alkyne attached to a second component such as the peptide. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The agent is prepared with the presence of either an alkyne (if the polymer contains an azide) or an azide (if the polymer contains an alkyne) group. The agent is then allowed to react with the nanocarrier via the 1,3-dipolar cycloaddition reaction with or without a catalyst which covalently couples the agent to the particle through the 1,4-disubstituted 1,2,3-triazole linker.

A thioether linker is made by the formation of a sulfur-carbon (thioether) bond in the form, for instance, of R₁—S—R₂. Thioether can be made by either alkylation of a thiol/mercaptan (—SH) group on one component such as the agent with an alkylating group such as halide or epoxide on a second component such as the nanocarrier. Thioether linkers can also be formed by Michael addition of a thiol/mercaptan group on one component such as an agent to an electron-deficient alkene group on a second component such as a polymer containing a maleimide group or vinyl sulfone group as the Michael acceptor. In another way, thioether linkers can be prepared by the radical thiol-ene reaction of a thiol/mercaptan group on one component such as an agent with an alkene group on a second component such as a polymer or nanocarrier.

A hydrazone linker is made by the reaction of a hydrazide group on one component such as the agent with an aldehyde/ketone group on the second component such as the nanocarrier.

A hydrazide linker is formed by the reaction of a hydrazine group on one component such as the agent with a carboxylic acid group on the second component such as the nanocarrier. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component such as the agent with an aldehyde or ketone group on the second component such as the nanocarrier.

An urea or thiourea linker is prepared by the reaction of an amine group on one component such as the agent with an isocyanate or thioisocyanate group on the second component such as the nanocarrier.

An amidine linker is prepared by the reaction of an amine group on one component such as the agent with an imidoester group on the second component such as the nanocarrier.

An amine linker is made by the alkylation reaction of an amine group on one component such as the agent with an alkylating group such as halide, epoxide, or sulfonate ester group on the second component such as the nanocarrier. Alternatively, an amine linker can also be made by reductive amination of an amine group on one component such as the agent with an aldehyde or ketone group on the second component such as the nanocarrier with a suitable reducing reagent such as sodium cyanoborohydride or sodium triacetoxyborohydride.

A sulfonamide linker is made by the reaction of an amine group on one component such as the agent with a sulfonyl halide (such as sulfonyl chloride) group on the second component such as the nanocarrier.

A sulfone linker is made by Michael addition of a nucleophile to a vinyl sulfone. Either the vinyl sulfone or the nucleophile may be on the surface of the nanocarrier or attached to the agent.

Any of the above linkers may comprise a reactive moiety as provided herein to couple a biologically active agent via the reactive moiety. Any of the above linkers may also be used to comprise other biologically active agents or other elements and may not comprise a reactive moiety as provided herein.

A biologically active agent, or nanocarrier element, can also be conjugated to the nanocarrier with non-covalent conjugation methods. For examples, a negative charged agent can be conjugated to a positive charged nanocarrier through electrostatic adsorption. A biologically active agent containing a metal ligand can also be conjugated to a nanocarrier containing a metal complex via a metal-ligand complex.

In embodiments, a biologically active agent, or other element, can be attached to a polymer via the reactive moiety, for example polylactic acid-block-polyethylene glycol, prior to the assembly of the synthetic nanocarrier or the synthetic nanocarrier can be formed with reactive or activatible groups on its surface. In the latter case, the agent may be prepared with a group which is compatible with the attachment chemistry that is presented by the synthetic nanocarriers' surface. In other embodiments, an agent can be attached to VLPs or liposomes using a suitable linker that may comprise a reactive moiety. A linker is a compound or reagent that capable of coupling two molecules together. In an embodiment, the linker can be a homobifuntional or heterobifunctional reagent as described in Hermanson 2008. For example, an VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with an agent containing an acid group via the other end of the ADH linker on NC to produce the corresponding VLP or liposome peptide conjugate.

For detailed descriptions of available conjugation methods, see Hermanson G T “Bioconjugate Techniques”, 2nd Edition Published by Academic Press, Inc., 2008. In addition to covalent attachment, the agent or other element can be coupled by adsorbtion to a pre-formed synthetic nanocarrier or it can be coupled by encapsulation during the formation of the synthetic nanocarrier.

### D. METHODS OF MAKING AND USING THE INVENTIVE COMPOSITIONS AND RELATED METHODS

Synthetic nanocarriers may be prepared using a wide variety of methods known in the art. For example, synthetic nanocarriers can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Various materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials, such as oligonucleotides, into synthetic nanocarriers may be used, including without limitation methods disclosed in U.S. Pat. No. 6,632,671 to Unger Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If particles prepared by any of the above methods have a size range outside of the desired range, particles can be sized, for example, using a sieve.

Elements of the inventive synthetic nanocarriers may be coupled to the overall synthetic nanocarrier, e.g., by one or more covalent bonds, or may be coupled by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be coupled to biologically active agents or other elements directly or indirectly via non-covalent interactions. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of an inventive synthetic nanocarrier. In embodiments, encapsulation and/or absorption is a form of coupling.

In embodiments, the inventive synthetic nanocarriers can be combined with other biologically active agents or other elements by admixing in the same vehicle or delivery system. Such biologically active agents may include immunomodulatory agents, such as mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIX™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments. The doses of such other biologically active agents or other elements can be determined using conventional dose ranging studies.

In embodiments, the inventive synthetic nanocarriers can also be combined with an immunomodulatory agent different, similar or identical to those coupled to a nanocarrier (with or without antigen, utilizing or not utilizing another delivery vehicle) administered separately at a different time-point and/or at a different body location and/or by a different immunization route or with another antigen and/or immunomodulatory agent-carrying synthetic nanocarrier administered separately at a different time-point and/or at a different body location and/or by a different immunization route.

Populations of synthetic nanocarriers may be combined to form pharmaceutical dosage forms according to the present invention using traditional pharmaceutical mixing methods. These include liquid-liquid mixing in which two or more suspensions, each containing one or more subset of nanocarriers, are directly combined or are brought together via one or more vessels containing diluent. As synthetic nanocarriers may also be produced or stored in a powder form, dry powder-powder mixing could be performed as could the re-suspension of two or more powders in a common media. Depending on the properties of the nanocarriers and their interaction potentials, there may be advantages conferred to one or another route of mixing.

Typical inventive compositions that comprise synthetic nanocarriers may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention comprise inventive synthetic nanocarriers in combination with pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, inventive synthetic nanocarriers are suspended in sterile saline solution for injection together with a preservative.

It is to be understood that the compositions of the invention can be made in any suitable manner, and the invention is in no way limited to compositions that can be produced using the methods described herein. Selection of an appropriate method may require attention to the properties of the particular moieties being associated.

In some embodiments, inventive synthetic nanocarriers are manufactured under sterile conditions or are terminally sterilized. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, inventive synthetic nanocarriers may be lyophilized and stored in suspension or as lyophilized powder depending on the formulation strategy for extended periods without losing activity.

The inventive compositions may be administered by a variety of routes of administration, including but not limited to subcutaneous, intramuscular, intradermal, oral, intranasal, transmucosal, sublingual, rectal, ophthalmic, transdermal, transcutaneous or by a combination of these routes.

Doses of dosage forms contain varying amounts of populations of synthetic nanocarriers and varying amounts of biologically active agents, according to the invention. The amount of synthetic nanocarriers and/or biologically active agents present in the inventive dosage forms can be varied according to the nature of the biologically active agents, the therapeutic benefit to be accomplished, and other such parameters. In embodiments, dose ranging studies can be conducted to establish optimal therapeutic amount of the population of synthetic nanocarriers and the amount of biologically active agents to be present in the dosage form. In embodiments, the synthetic nanocarriers and immunomodulatory agents are present in the dosage form in an amount effective to modulate an immune response upon administration to a subject. It may be possible to determine amounts of the immunomodulatory agents effective to modulate an immune response using conventional dose ranging studies and techniques in subjects. Inventive dosage forms may be administered at a variety of frequencies. In a preferred embodiment, at least one administration of the dosage form is sufficient to generate a pharmacologically relevant response. In more preferred embodiment, at least two administrations, at least three administrations, or at least four administrations, of the dosage form are utilized to ensure a pharmacologically relevant response.

The compositions and methods described herein can be used to modulate (e.g., induce, enhance, suppress, direct, redirect, etc.) an immune response. The compositions and methods described herein can be used in the diagnosis, prophylaxis and/or treatment of conditions such as cancers, infectious diseases, metabolic diseases, degenerative diseases, autoimmune diseases, inflammatory diseases, immunological diseases, or other disorders and/or conditions. The compositions and methods described herein can also be used for the prophylaxis or treatment of an addiction, such as an addiction to nicotine or a narcotic. The compositions and methods described herein can also be used for the prophylaxis and/or treatment of a condition resulting from the exposure to a toxin, hazardous substance, environmental toxin, or other harmful agent.

### E. EXAMPLES

### Example 1

### Synthesis of a Thiol-Reactive and Hydride Reductive Benzoquinone

### Step 1.

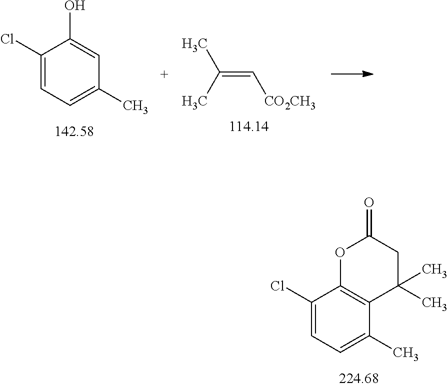

A solution of 2-chloro-5-methylphenol (28.5 gm, 0.20 moles) and methyl 3,3-dimethylacrylate (25.1 gm, 0.22 moles) in methanesulfonic acid (125 mL) was stirred and heated at 70° C. for 5 hours. After cooling, the solution was poured into ice and water (300 gm), and the precipitated oil was extracted into t-butylmethyl ether (TBME, 150 mL). The TBME solution was diluted with hexane (100 mL), and the resulting solution was extracted with 5% potassium hydroxide solution (2×50 mL). Some product began to crystallize at this point, and ethyl acetate (100 mL) was added to keep the lactone in solution. The organic solution was dried (MgSO₄), filtered and evaporated under vacuum. The remaining solid was recrystallized from ethyl acetate (25 mL) and hexane (100 mL) to give the lactone (4,4,5-trimethyl-8-chloro-3,4-dihydro-1-benzopyran-2-one) as a white crystalline solid in a yield of 10.6 gm (23.6%).

### Step 2.

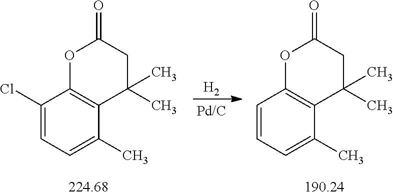

The lactone (2.24 gm, 0.01 moles) and potassium acetate (1.96 gm, 0.02 moles) were combined with 10% palladium on carbon (400 mg containing 50% water) in acetic acid (25 mL). This mixture was hydrogenated at 40 PSI of hydrogen on a Parr hydrogenation apparatus. After 5 hours, hydrogen consumption had stopped and the reaction mixture was filtered through a pad of diatomaceous earth. The clear filtrates were evaporated under vacuum at 60° C. and to the residue was added water (100 mL). The solid which formed was extracted into ethyl acetate (200 mL), and this solution was washed with saturated sodium bicarbonate solution (50 mL).

The organic solution was dried (Na₂SO₄), filtered and evaporated under vacuum to provide the dechlorinated lactone (4,4,5-trimethyl-3,4-dihydro-1-benzopyran-2-one) as a white solid. The yield was quantitative.

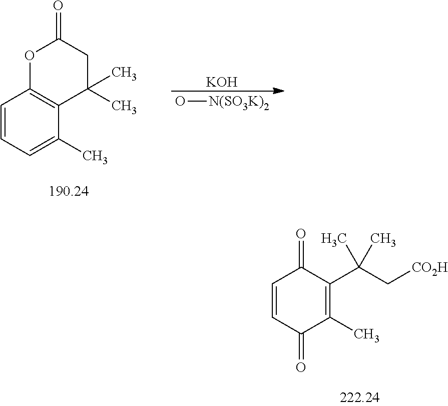

### Step 3.

A portion of the lactone (2.53 gm, 1.33×10⁻³ moles) was warmed in methanol (15 mL) and water (15 mL) containing potassium hydroxide (1.92 gm, 85%, 2.90×10⁻² moles). Once dissolved, the solution was diluted with water (50 mL).

Disodium hydrogen phosphate dihydrate (1.95 gm, 1.1×10⁻² moles) was dissolved in water (300 mL), and to this solution was added potassium nitrosodisulfonate (9.0 gm, 3.3×10⁻² moles). After stirring for 10 minutes the solution of the hydrolyzed lactone, from above, was added.

The reaction was allowed to stir for 30 minutes after which the reaction was extracted with t-butylmethyl ether (TBME, 100 mL). The pH of the aqueous was adjusted to 1.0 with phosphoric acid, and after stirring for 10 minutes, the aqueous reaction mixture was extracted with methylene chloride (3×150 mL). The combined yellow extracts were dried (Na₂SO₄), filtered and evaporated under vacuum to give the quinone as a bright yellow solid in a yield of 2.16 gm (73%).

### Step 4.

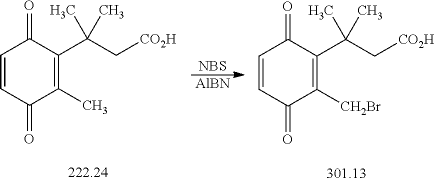

To a solution of the lactone (1.29 gm, 5.80×10⁻³ moles) in carbon tetrachloride (20 mL) was added N-bromosuccinimide (NBS, 1.3 gm, 8.96×10⁻³ moles). This mixture was warmed and azo bis-isobutyronitrile (AIBN, 0.20 gm) was added. The reaction was stirred at reflux for 4 hours. The solvent was removed under vacuum, and the product was purified by chromatography on silica using 5% methanol in methylene chloride as eluent. The bromomethyl quinone was isolated in a yield of 1.57 gm (90%).

### Step 5.

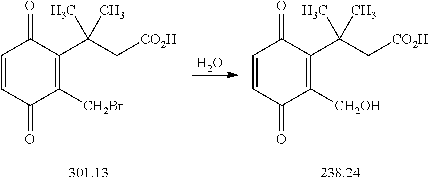

The bromomethyl quinone (1.57 gm, 5.2×10⁻³ moles) was stirred in water (30 mL) and dioxane (20 mL), and this mixture was stirred at reflux for one hour. After cooling, the reaction was diluted with water (300 mL), and this mixture was extracted with methylene chloride (3×100 mL). The combined extracts were dried (Na₂SO₄), filtered and evaporated under vacuum to provide the crude lactone. The product was purified by chromatography on silica using 5% methanol in methylene chloride as eluent. The hydroxymethyl quinone was isolated in a yield of 0.87 gm (70%).

### Step 6.

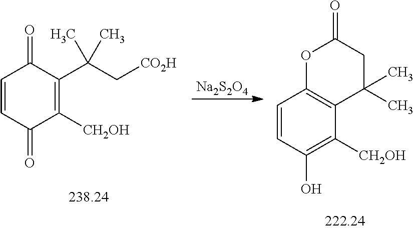

A solution of the quinone (0.87 gm, 3.6×10⁻³ moles) in t-butylmethyl ether (TBME, 100 mL) was shaken in a separatory funnel with a solution of sodium hydrosulfide (5 gm) in water (50 mL) until the color faded from yellow to colorless. The TBME solution was isolated, washed with water (50 mL) and the organic solution was dried (Na₂SO₄), filtered and evaporated under vacuum to provide the lactone as a white solid. The yield was quantitative.

### Step 7.

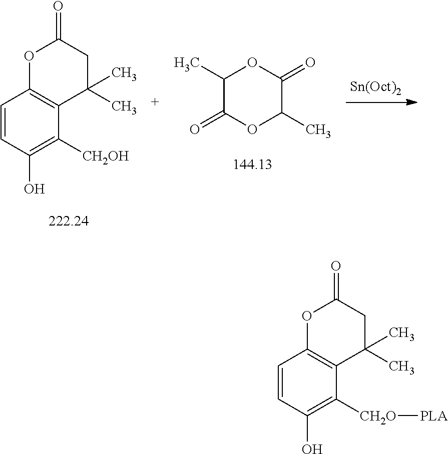

A mixture of the lactone (0.333 gm, 1.5×10⁻³ moles) and D/L lactide (10.5 gm, 7.28×10⁻² moles) in toluene (100 mL) was heated to reflux and a portion of the toluene (50 mL) was distilled to dry the reaction solution. After cooling slightly under argon, tin (II) ethylhexanoate (200 μL) was added and the solution was heated at 120° C. for 16 hours. After cooling, the toluene was evaporated under vacuum, and the remaining polymer mass was dissolved in ethyl acetate (100 mL). To this solution was added a solution of ethylenediamine tetraacetic acid tetrasodium salt (10 gm) in water (10 mL). After stirring vigorously for 2 minutes, acetic acid (3.2 mL) was added, and vigorous stirring was continued. After about 15 minutes the EDTA, water and acetic acid solidified and formed precipitate in the flask. The ethyl acetate was decanted from the solid and was dried over sodium sulfate. After filtration, the ethyl acetate was removed under vacuum and the polymer was dried under high vacuum. NMR confirmed the structure and indicated a molecular weight of about 7 KD. The yield was 9.0 gm.

### Step 8.

The lactone polymer (9.0 gm, 1.29×10⁻³ moles) was dissolved in acetonitrile (50 mL) and water (3 mL). This solution was stirred as N-bromosuccinimide (NBS, 237 mg, 1.33×10⁻³ moles) dissolved in acetonitrile (5 mL) was slowly added. Upon addition of the NBS, the colorless solution turned bright yellow. After stirring for 10 minutes, the solution was diluted with ethyl acetate (300 mL), and this solution was washed with water (2×100 mL). The ethyl acetate solution was dried over sodium sulfate, then filtered and evaporated under vacuum to a volume of 75 mL. With stirring, 2-propanol (300 mL) was slowly added which caused the polymer to precipitate as a yellow mass. The 2-propanol was decanted from the polymer, and excess 2-propanol was removed under vacuum. After drying under high vacuum there was obtained 7.5 gm of polymer.

### Example-2

### Synthesis of a Thiol-Reactive and Hydride-Reductive Benzoquinone-Polymer Conjugate

### Step 1.

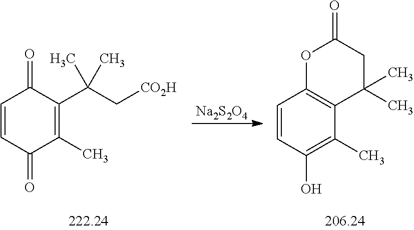

A solution of the quinone (2.22 gm, 1.0×10⁻² moles) in t-butylmethyl ether (TBME, 200 mL) was shaken in a separatory funnel with a solution of sodium hydro sulfide (10 gm) in water (100 mL) until the color faded from yellow to colorless. The TBME solution was isolated, washed with water (100 mL) and the organic solution was dried (Na₂SO₄), filtered and evaporated under vacuum to provide the lactone as a white solid. The yield was quantitative.

### Step 2.

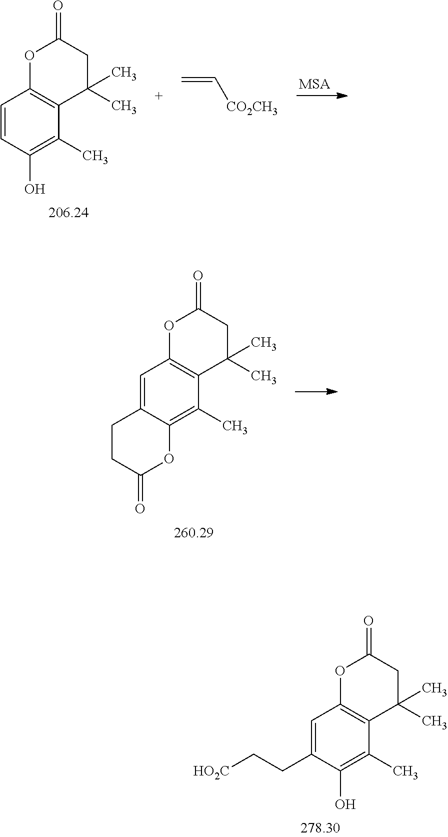

The lactone (2.68 gm, 0.013 moles) and methyl acrylate (1.25 gm, 1.3 mL, 0.014 moles) were combined in methanesulfonic acid (20 mL), and the resulting solution was heated at 70° C. overnight. After cooling, the solution was poured onto ice (500 gm). The precipitated oil was extracted into ethyl acetate (200 mL). This solution was washed with water (100 mL), dried (MgSO₄), filtered and evaporated under vacuum. The residue was dissolved in a combination of THF (50 mL) and 2-propanol (50 mL). This solution was stirred and de-aerated with argon as a solution of potassium hydroxide (5 gm), and sodium borohydride (100 mg) in water (25 mL) was slowly added. The resulting pale yellow solution was stirred for 30 minutes and was then acidified with concentrated hydrochloric acid. Water (300 mL) was added, and the reaction was extracted with ethyl acetate (200 mL). This solution was then extracted with saturated sodium bicarbonate solution (2×50 mL) and water (50 mL). The combined aqueous extracts were acidified with concentrated hydrochloric acid, and the precipitated oil was extracted into ethyl acetate (200 mL). This solution was dried (MgSO₄), filtered and evaporated under vacuum. The residue was triturated in diethyl ether, and the solid product was isolated by filtration and dried. The yield was 543 mg (15%).

### Step 3.

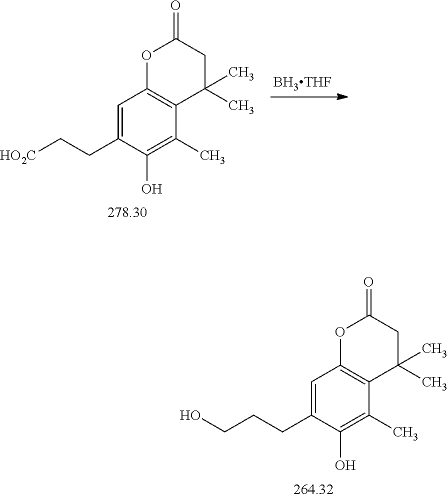

The acid (0.557 gm, 2×10⁻³ moles) was dissolved in dry THF (10 mL) under argon. This solution was stirred as borane in THF (4.0 mL, 1.0 M, 4×10⁻³ moles) was added via syringe in 10 minutes. The resulting solution was stirred at room temperature for 2 hours. To the reaction was added 20% hydrochloric acid (10 mL), and stirring was continued for 30 minutes. The reaction was partitioned between water (50 mL) and ethyl acetate (100 mL), and the ethyl acetate solution was washed with saturated sodium bicarbonate (50 mL) and then brine (50 mL). This solution was dried (Na₂SO₄), filtered and evaporated under vacuum. The solid residue was purified by chromatography on silica using 5% methanol in methylene chloride as eluent. There was obtained 0.40 gm (76%) of the purified product as a white solid.

### Step 4.

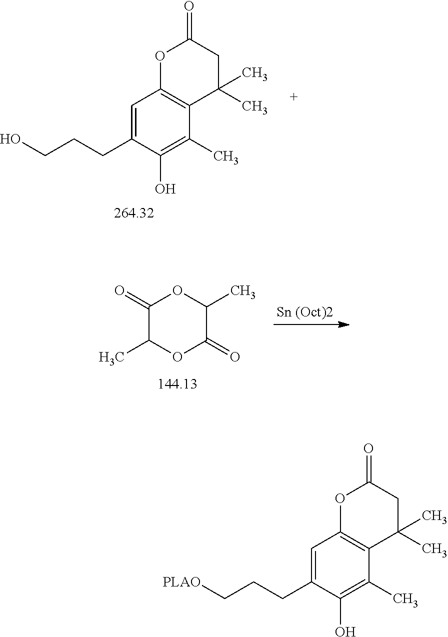

A mixture of the lactone (0.362 gm, 1.37×10⁻³ moles) and D/L lactide (9.6 gm, 6.65×10⁻² moles) in toluene (100 mL) was heated to reflux, and a portion of the toluene (50 mL) was distilled to dry the reaction solution. After cooling slightly under argon, tin (II) ethylhexanoate (200 μL) was added, and the solution was heated at 120° C. for 16 hours. After cooling, the toluene was evaporated under vacuum, and the remaining polymer mass was dissolved in methyl acetate. To this solution was added a solution of ethylenediamine tetraacetic acid tetrasodium salt (10 gm) in water (10 mL). After stirring vigorously for 2 minutes, acetic acid (3.2 mL) was added and vigorous stirring was continued. After about 15 minutes, the EDTA, water and acetic acid solidified and formed precipitate in the flask. The methyl acetate was decanted from the solid and was dried over sodium sulfate. After filtration, the methyl acetate was removed under vacuum, and the polymer was dried under high vacuum. NMR confirmed the structure and indicated a molecular weight of 7 KD. The yield was 9.5 gm. Tin analysis showed that the polymer contained about 5 ppm of tin.

### Step 5.

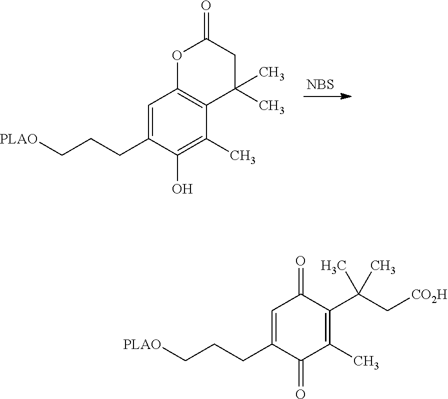

The lactone polymer (9.5 gm, 1.36×10⁻³ moles) was dissolved in acetonitrile (50 mL) and water (3 mL). This solution was stirred as N-bromosuccinimide (NBS, 250 mg, 1,40×10⁻³ moles) dissolved in acetonitrile (5 mL) was slowly added. Upon addition of the NBS, the colorless solution turned bright yellow. After stirring for 10 minutes, the solution was diluted with ethyl acetate (300 mL), and this solution was washed with water (2×100 mL). The ethyl acetate solution was dried over sodium sulfate, then filtered and evaporated under vacuum to a volume of 75 mL. With stirring, 2-propanol (300 mL) was slowly added which caused the polymer to precipitate as a yellow mass. The 2-propanol was decanted from the polymer and excess 2-propanol was removed under vacuum. After drying under high vacuum there was obtained 8.0 gm of polymer.

### Example 3

### Synthesis of a Thiol-Reactive and Hydride-Reductive Quinone-Immunomodulatory Agent Conjugate

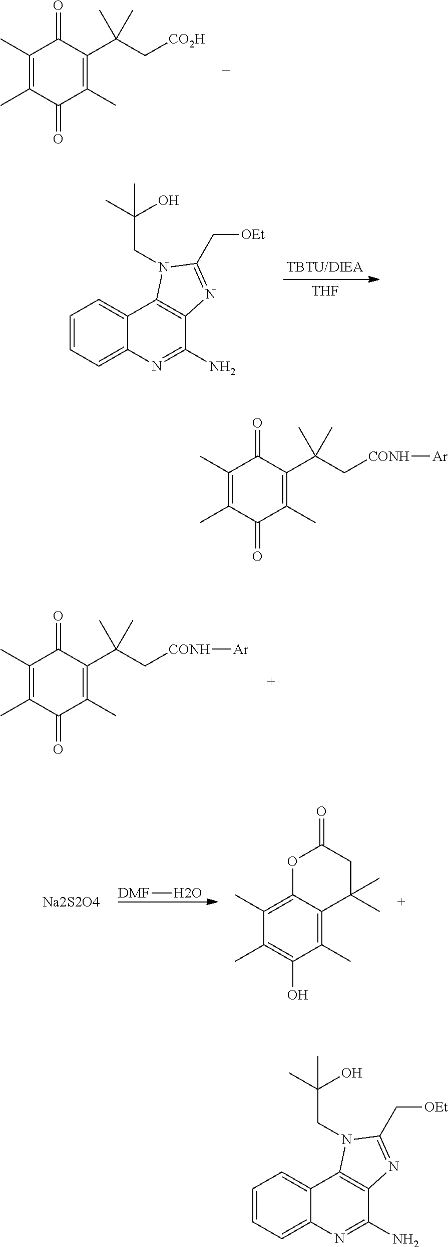

Benzoquinone acid (0.751 g, 3 mmol) and TBTU (0.963 g, 3.0 mmol) were dissolved in 30 mL of dry THF. R848 (0.628 g, 2 mmol) was added, followed by DIEA (1.5 mL, 9 mmol). The resulting mixture was stirred at room temperature overnight. The mixture was diluted with EtOAc (100 mL) and washed with water, NH₄Cl and brine (20 mL each). After drying over Na₂SO₄, the solution was concentrated to give a brown solid which was purified by recrystallization from EtOAc/MTBE to give the desired amide as a light brown solid (0.47 g, 43% yield).

The amide (20 mg) was dissolved in DMF (1 mL) and water (0.5 mL). A solution of Na₂S2O₄ (sodium hydrosulfite) (25 mg) in 0.2 mL of water was added. The resulting mixture was stirred at room temperature for 4 h. TLC showed the disappearance of the amide and appearance of R848 and the hydroquinone lactone.

### Example 4

### Preparation of Polymers with a Thiol End Group

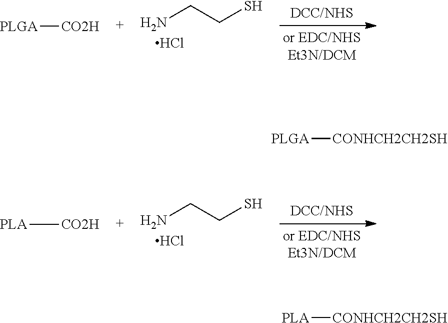

A mixture of PLGA-CO₂H (Lakeshores Polymers, 7525DLG1A, acid number 0.46 mmol/g, 5.0 g, 2.3 mmol, 1.0 eq), EDC (1.07 g, 6.9 mmol), N-hydroxysuccinimide (NHS) (0.79 g, 6.9 mmol), cysteamine hydrochloride (0.79 g, 6.9 mmol) and Et₃N (1.9 mL, 13.8 mmol) in DCM (50 mL) was stirred at room temperature under argon for 2 days. The mixture was then concentrated to a residual. Isopropyl alcohol (IPA) (100 mL) was added to precipitate the polymer. The polymer was then washed with water (2×50 mL), IPA (50 mL) and MTBE (50 mL) and dried under vacuum to give PLGA-cysteamine conjugate as a white foamy solid (5.8 g, H NMR in CDCl₃ confirms the product).

In a similar fashion, PLA-cysteamine conjugate can be prepared from PLA-CO₂H.

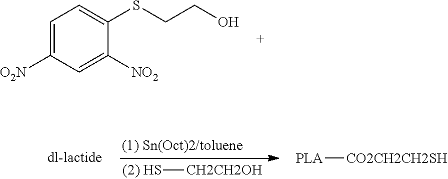

2-(2,4-Dinitrophenylthio)ethanol was prepared according to the literature procedure ((G. Carrot and J. G. Hilborn, et al; Macromolecules 1999, 32, 5264-5269) with minor modification. 2-Mercaptoethanol (6.91 mL, 100 mmol) in 80 mL of DCM was slowly added to a solution of 2,4-dinitrofluorobenzene (18.6 g, 100 mmol) in 28 mL (200 mmol) of triethylamine under ice water cooling. The resulting thick yellow slurry was then stirred at room temperature overnight, and a brown solution was formed. The solution was diluted with DCM (200 mL) and washed with water (50 mL), saturated NH₄Cl (50 mL) and saturated NaCl (50 mL). After drying over Na₂SO₄, the solution was concentrated to give a dark brown oil. The brown oil was recrystallized in EtOAc/MTBE to give 2-(2,4-Dinitrophenylthio)ethanol as a yellow solid (13.2 g, 54% yield).

To polymerize 2-(2,4-Dinitrophenylthio)ethanol with dl-lactide a 100 mL flask equipped with a stir bar and an azeotrope condenser was charged with dl-lactide (14.4 g, 100 mmol), 2-(2,4-Dinitrophenylthio)ethanol from above (0.25 g, 1.0 mmol) and dry toluene (80 mL). The mixture was heated to reflux while about 40 mL of toluene was distilled out. The brownish solution was then cooled to ca. 110° C. (oil bath temperature), and Sn(Oct)₂ (0.32 mL, 1.0 mmol) was added to the solution. The resulting solution was heated at reflux overnight (16 h) and cooled to room temperature. The solution was then added to 200 mL of IPA to precipitate out the brown colored polymer. The polymer was washed with 50 mL of IPA and 50 mL of MTBE and dried at 30° C. under vacuum as a brown foamy solid (14 g).

To prepare poly-dl-lactide (PLA) with a thiol end group, the polymer from above was dissolved in 30 mL of DCM, and 2-mercaptoethanol (7.8 mL, 100 mmol) was added. The pH of the solution was adjusted to 8 with Et₃N. The resulting orange solution was stirred at room temperature overnight (20 h) and then added to 200 mL of IPA to precipitate out PLA-CO₂CH₂CH₂SH. The polymer was then washed with 50 mL of IPA and 50 mL of MTBE and dried at 30° C. under vacuum (13 g, MW ca. 14000 by NMR and GPC).

### Example 5

### Preparation of a Polymer Disulfide Linked Immunomodulatory Agent (R848)

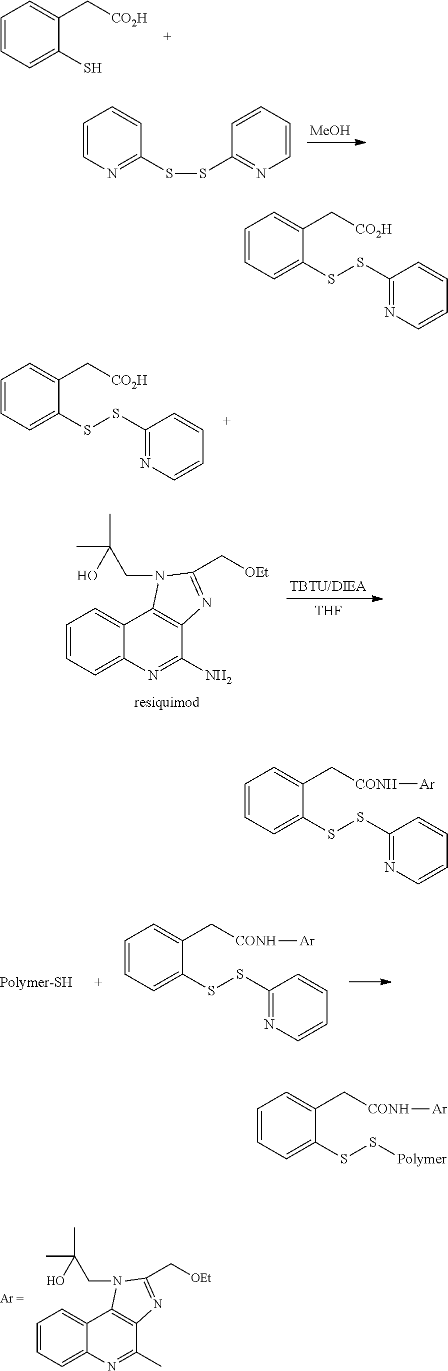

2-sulfhydrylphenylacetic acid was prepared from benzothiophene-2-boronic acid by hydrogen peroxide oxidation to benzothiophene-2-one, followed by hydrolysis with lithium hydroxide as described in the literature (Shuyi Chen, et al; Bioconjugate Chem. 2010, 21, 979-987). Hydrogen peroxide (20 mL, 30% wt aq) was added to a solution of benzothiophene-2-boronic acid (10 g, 56.2 mmol) in 100 mL of EtOH with cooling. The resulting solution was stirred at room temperature for 18 h. The solution was then diluted with water (100 mL) and extracted with 2×100 mL of DCM. The organic phase was then washed with water (50 mL), NaHCO₃ (50 mL) and brine (50 mL) and dried over Na₂SO₄. After removal of DCM, the residual was dissolved in THF-water (50 mL each) and LiOH monohydrate (14 g, 336 mmol) was added. The resulting orange solution was heated at 60° C. overnight. The solution was cooled with ice water, diluted with 100 mL of MTBE and carefully acidified to pH 2 with concentrated HCl. The phases were separated and the aqueous phase was extracted with 100 mL of MTBE. The combined MTBE phase was washed with NaCl and dried over Na₂SO₄. After concentration, the resulting orange solid was recrystallized from DCM-hexanes to give golden solid as 2-sulfhydrylphenylacetic acid (6.6 g, 68% yield).

To prepare 2-pyridine-2-yl-disulfanylphenylacetic acid, a solution of 2-sulfhydrylphenylacetic acid (5.1 g, 29.5 mmol) and 2-pyridine disulfide (7.2 g, 32.5 mmol) in 100 mL of MeOH was stirred at room temperature for 16 h. The solution was then concentrated to dryness. The residual was then purified on silica gel eluting with 5-10% MeOH in DCM to give 2-pyridine-2-yl-disulfanylphenylacetic acid (8.0 g, 95% yield).

To prepare 2-pyridine-2-yl-disulfanylphenylacetamide of R848,2-pyridine-2-yl-disulfanylphenylacetic acid (85 mg, 0.3 mmol) was dissolved in 10 mL of dry THF. TBTU (O-(Benzotriazol-1-yl)-N,N,N′,N′-tetramethyluronium tetrafluoroborate) (161 mg, 0.5 mmol) was added. The mixture was stirred at room temperature under argon for 10 min. R848 (80 mg, 0.25 mmol) was added, followed by DIPEA (diisopropylethylamine) (0.174 mL, 1.0 mmol). The resulting mixture was stirred at room temperature overnight. The reaction solution was then diluted with EtOAc (70 mL) and washed with water, saturated NH₄Cl and brine and dried over Na₂SO₄. The crude product was purified on silica gel to give the desired product (ca. 80 mg).

The polymer disulfide conjugate was prepared by thiol-disulfide exchange reaction between the polymer with thiol end group and 2-pyridine-2-yl-disulfanylphenylacetamide of R848. PLGA-cysteamine (300 mg) was dissolved in THF and 2-pyridine-2-yl-disulfanylphenylacetamide of R848 (40 mg) was added. The solution was stirred at room temperature overnight. The solution was then added to 30 mL of 2-propanol to precipitate out the polymer. The supernatant containing excess reactants and byproduct was removed. The polymer was washed with 2-propanol and MTBE and dried at room temperature under vacuum to give PLGA-disulfide-linked R848.

### Example 6

### Preparation of a Polymer Disulfide Linked Immunomodulatory Agent (8-Oxoadenine)

### Step 1.

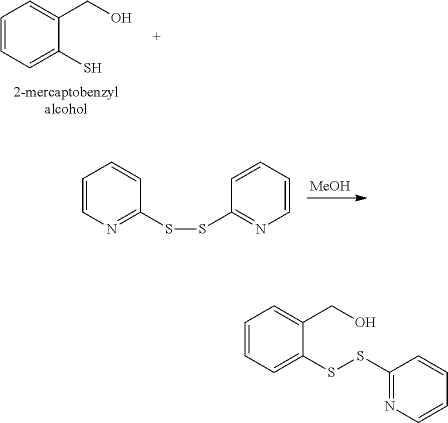

2-mercaptobenzyl alcohol (2.0, 14.3 mmol) and acetic acid (0.9 mL, 14.3 mmol) were dissolved in 30 mL of MeOH. 2,2′-Dithiopyridine (2,2′-dipyridyl disulfide) (3.14 g, 14.3 mmol) was added. The resulting yellow solution was stirred at room temperature overnight. The solution was concentrated to dryness and ethyl ether was added to the residual. The resulting precipitate (2-mercaptopyridine) was removed by filtration, and the filtrate was concentrated to give the crude product which was used without further purification (ca. 4.1 g, liquid).

### Step 2.

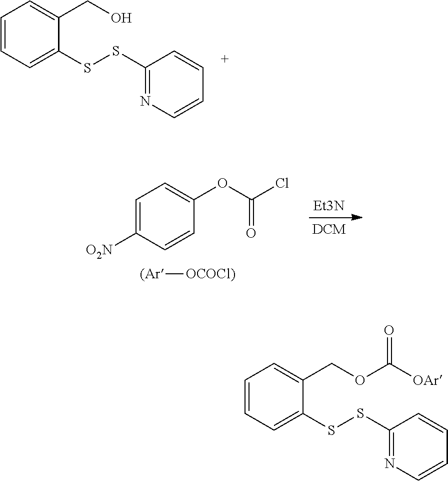

Crude 2′-pyridyl dithio-benzyl alcohol (2.49 g, 10 mmol) from above was dissolved in 50 mL dry DCM. The solution was cooled with ice water. Et₃N (2.8 mL, 20 mmol) was added. A solution of p-nitrobenzene chloroformate (2.0 g, 10 mmol) in DCM (10 mL) was added dropwise. The resulting mixture was stirred at room temperature overnight. The mixture was then concentrated to dryness, and the crude product was purified on silica gel eluting with DCM to give the 2′-pyridyl dithio-benzylcarbonate as a dense liquid (1.77 g).

### Step 3.

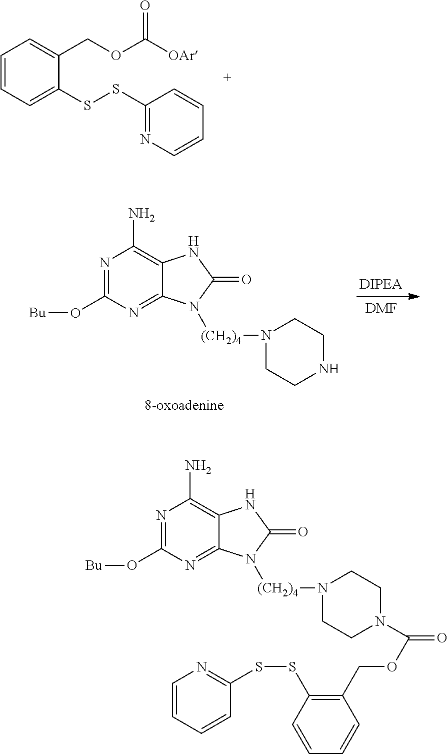

2′-pyridyl dithio-benzylcarbonate (124 mg, 0.3 mmol, 1.0 eq) from above is dissolved DMF (5 mL) and DIPEA (0.22 mL, 1.25 mmol, 5.0 eq) is added, followed by the 8-oxoadenine (prepared according to WO2010/018131) (1.0 eq). The mixture is stirred at room temperature overnight. The mixture is diluted with EtOAc and washed with water, brine and dried over Na2SO4. The crude product is purified on silica gel to give desired product.

### Step 4.

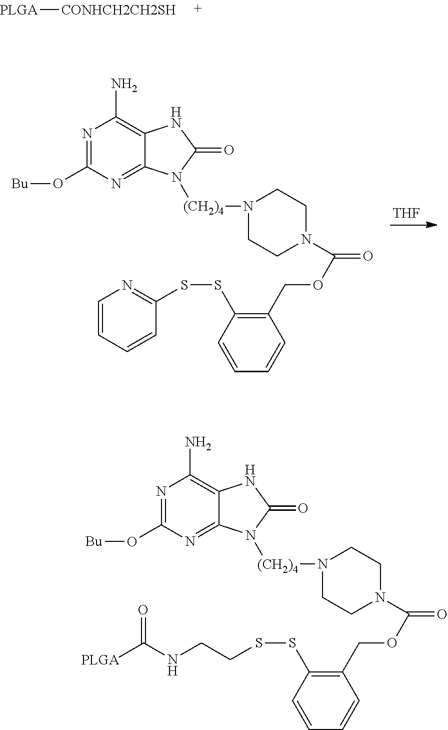

A mixture of PLGA-cysteamine and the pyridyl dithio-8-oxoadenine from above in THF is stirred at room temperature overnight. The solution is then added to 2-propanol to precipitate out the polymer conjugate. The supernatant is removed, and the residual polymer is washed with 2-propanol and MTBE and dried to give the polymer-disulfide-linked 8-oxoadenine conjugate.

### Example 7

### Synthesis of a Menadione Analog Conjugated to N-methyl piperazine

### Step 1.

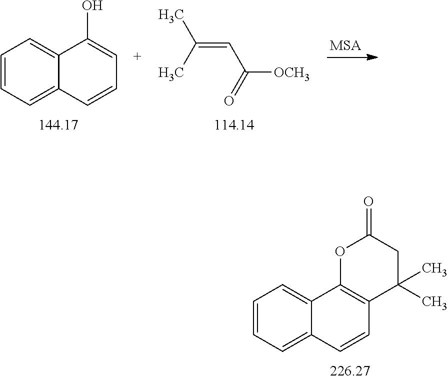

A solution 1-naphthol (28.8 gm, 0.20 moles) and methyl 3,3-dimethylacrylate (25.1 gm, 0.22 moles) in methanesulfonic acid (125 mL) was stirred and heated at 70° C. for 2 hours and then overnight at room temperature. After cooling, the solution was poured into ice and water (600 gm), and the precipitated oil was extracted into ethyl acetate (300 mL). The ethyl acetate solution was evaporated under vacuum, and the oily residue was stirred with methanol (100 mL) and water (100 mL). To this was added potassium hydroxide pellets (33 gm) and stirring was continued for 45 minutes. Water (300 mL) was added and an insoluble oil was extracted into diethyl ether (2×100 mL)¹. The aqueous was strongly acidified with phosphoric acid to a pH of about 1.0, and the mixture was heated at 70° C. for 30 minutes to promote lactonization. After cooling the oil which had separated was extracted into t-butyl ether (TBME, 300 mL). This solution was extracted with 5% potassium hydroxide solution (2×100 mL) and then with 10% phosphoric acid (100 mL). After washing with water (100 mL), the organic solution was dried (MgSO₄), filtered and evaporated under vacuum. The remaining solid was recrystallized from ethyl acetate (25 mL) and heptane (75 mL) to give the lactone in a yield of 23.1 gm (51%).
1. After drying over magnesium sulfate followed by filtration and evaporation of the solvent, the residue was crystallized from heptane to provide the cyclic ether in a yield of 6.8 gm, (15%). The structure of the cyclic ether is shown below.

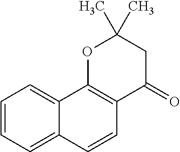

### Step 2.

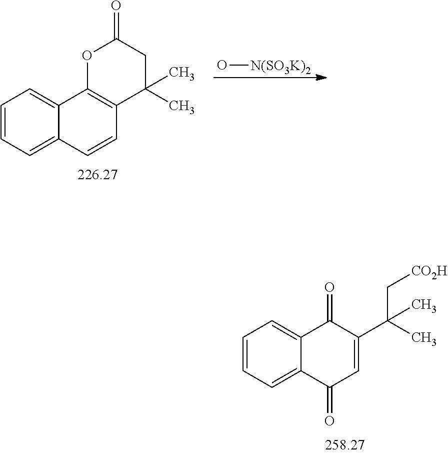

A portion of the lactone (3.5 gm, 1.55×10⁻² moles) was warmed in methanol (15 mL) and water (15 mL) containing potassium hydroxide (2.24 gm, 85%, 3.39×10⁻² moles). Once dissolved, the solution was diluted with water (75 mL).

Sodium dihydrogen phosphate dihydrate (2.28 gm) was dissolved in water (300 mL) and to this solution was added potassium nitrosodisulfonate (10.5 gm, 3.85×10⁻² moles). After stirring for 10 minutes the solution of the hydrolyzed lactone, from above, was added.

The reaction was allowed to stir for 30 minutes after which the pH of the aqueous was adjusted to 1.0 with phosphoric acid and then the aqueous reaction mixture was extracted with methylene chloride (2×175 mL). The combined yellow extracts were dried (Na₂SO₄), filtered and evaporated under vacuum to give the quinone as a bright yellow solid in a yield of 2.9 gm (73%).

### Step 3.

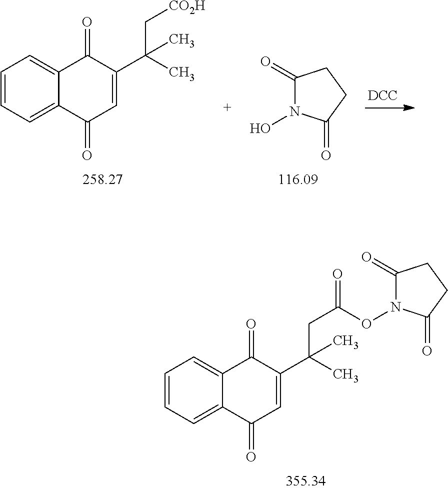

The quinone (700 mg, 2.7×10⁻³ moles), N-hydroxysuccinimide (345 mg, 3.0×10⁻³ moles), and dicyclohexylcarbodiimide (619 mg, 3.0×10⁻³ moles) were dissolved in a mixture of dimethyl formamide (5 mL) and tetrahydrofuran (10 mL). The orange solution was stirred overnight at room temperature and was then diluted with diethyl ether (200 mL). This was washed with 5% phosphoric acid (100 mL) followed by saturated sodium bicarbonate (100 mL). After drying over magnesium sulfate, the solution was filtered and evaporated under vacuum. The residual yellow solid was purified by chromatography on silica using 5% methanol in methylene chloride as eluent. Evaporation of the solvents gave the amide in a yield of 655 mg (68%).

### Step 4.

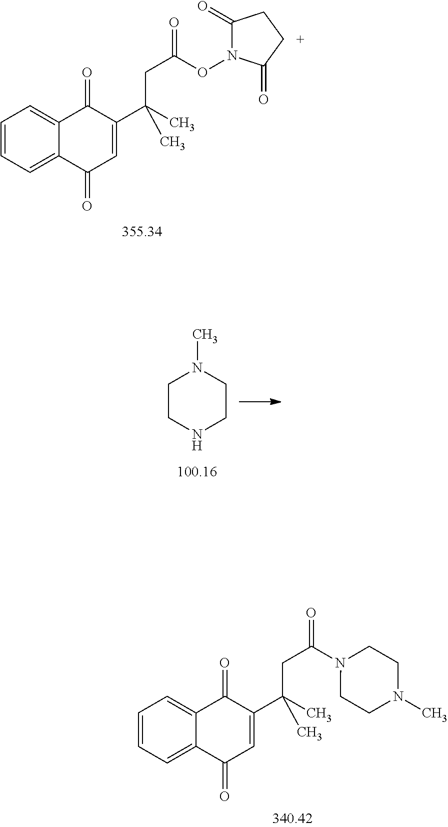

The quinone NHS ester (600 mg, 1.69×10⁻³ moles) was dissolved in a mixture of dichloroethane (20 mL) and dimethylformamide (5 mL). To this solution was added n-methylpiperazine (186 mg, 206 μL, 1.86×10⁻³ moles) and 4-dimethylaminopyridine (50 mg). This solution was kept at 50° C. for 5 hours. After cooling the reaction was diluted with ethyl acetate (100 mL), and the resulting solution was washed with 5% phosphoric acid (50 mL) followed by saturated sodium bicarbonate (50 mL). After drying over magnesium sulfate, the solution was filtered and evaporated under vacuum. The residue was purified by chromatography on silica using 5% methanol in methylene chloride as eluent. Evaporation of the solvents gave the NHS ester in a yield of 404 mg (44%).

### Example 8

### Release by Thiol Addition and Hydride Reduction

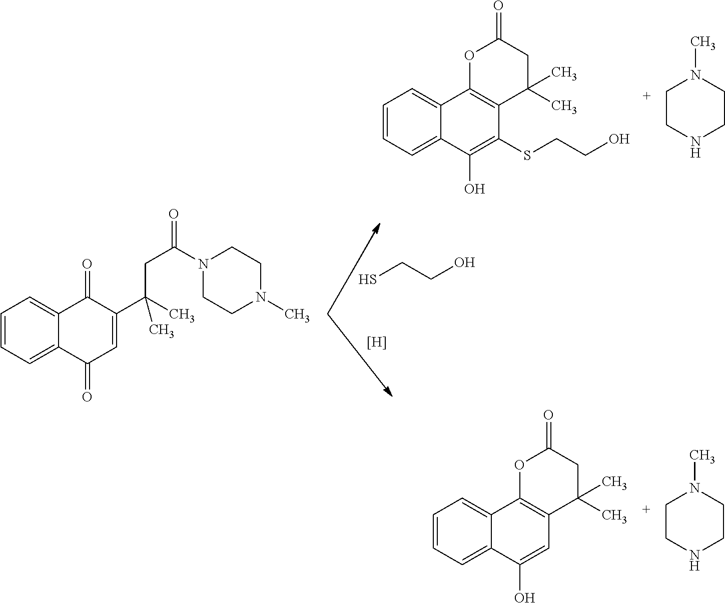

A portion of the amide was dissolved in water with a drop of acetic acid. This yellow solution was divided into two portions. One portion was treated with sodium dithionite, a reducing agent, and the other was treated with 2-mercaptoethanol. After 2 hours, the samples were diluted with water and extracted with ethyl acetate. TLC of the extracts with 50/50 ethyl acetate/hexane as eluent clearly showed that, in both cases, the amide had been consumed with a colorless, non-polar product (lactone) being formed.

### Example 9

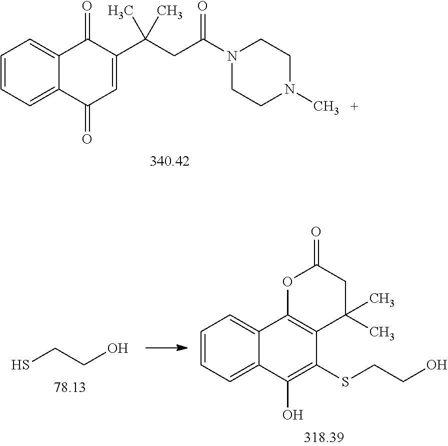

The quinone amide (518 mg, 1.52×10⁻³ moles) was dissolved in water containing acetic acid (200 mg, 3.3×10⁻³ moles). To this solution was added 2-mercaptoethanol (238 mg, 3.04×10⁻³ moles). After stirring at room temperature for 48 hours, a TLC (silica, 10% methanol in methylene chloride) of the solution showed a single product had formed. The reaction was diluted with water (20 mL) and this mixture was extracted with methylene chloride (3×20 mL). The combined extracts were washed with saturated sodium bicarbonate solution and then dried over sodium sulfate. After filtration to remove the drying agent, the methylene chloride was evaporated under vacuum to give the product as an oil. This was purified by chromatography on silica using 5% methanol in methylene chloride as eluent. The fractions that contained the product were pooled and evaporated under vacuum. NMR of the product confirmed that the material was the mercaptoethanol adduct of the lactone.

### Example 10

### Synthesis of a Thiol-reactive and Hydride-reductive Orthoquinone

### Step 1.

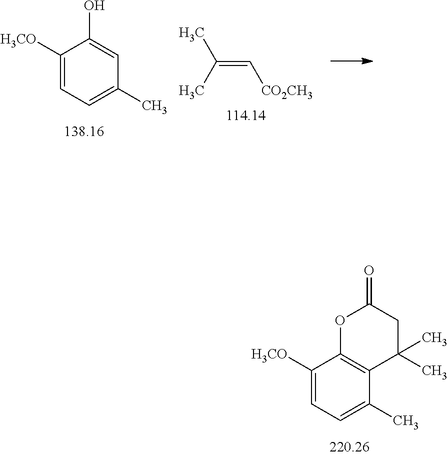

A solution of 2-methoxy-5-methylphenol (10 gm, 0.072 moles) and methyl 3,3-dimethylacrylate (9.09 gm, 0.08 moles) in methanesulfonic acid (50 mL) was stirred and heated at 70° C. for 2 hours. After cooling, the solution was poured into ice and water (500 gm), and the precipitated material was extracted into 50/50 ethyl acetate and hexane (400 mL). This solution was washed with 5% potassium hydroxide solution (2×100 mL) followed by 5% phosphoric acid (100 mL). The organic solution was dried (MgSO₄), filtered and evaporated under vacuum. The remaining white solid was recrystallized from heptanes containing a small amount of ethyl acetate to give the lactone as a white crystalline solid in a yield of 9.4 gm (59.3%).

### Step 2.

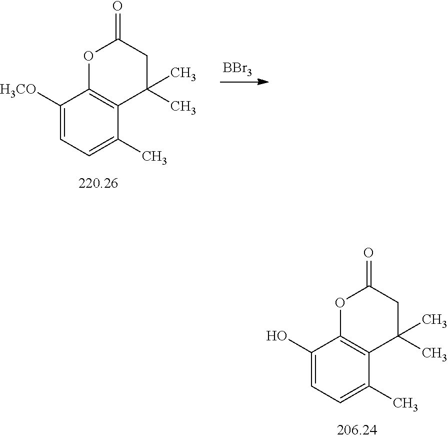

A solution of the methoxy lactone (4.4 gm, 2.0×10⁻² moles) in dichloromethane (40 mL) was stirred under argon and cooled in a dry ice/2-propanol bath. With stirring, a solution of boron tribromide (10 gm, 3.84 mL, 4.0×10⁻² moles) in dichloromethane (20 mL) was added dropwise. Once addition was complete, the solution was stirred at room temperature overnight. The clear solution was diluted with dichloromethane (100 mL), and this solution was cautiously treated with water. After stirring for 30 minutes, the dichloromethane layer was isolated and washed with saturated sodium bicarbonate solution. After drying over sodium sulfate, the solution was filtered and evaporated under vacuum. The residual oil was crystallized from 5% ethyl acetate/heptanes to give the product as a white crystalline solid in a yield of 3.5 gm (84.9%).

### Step 3.

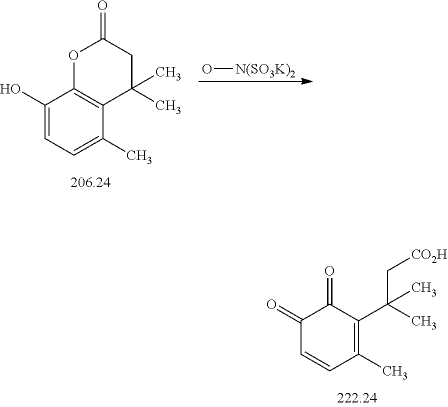

A portion of the hydroxylactone (2.06 gm, 0.01 moles) was dissolved in methanol (15 mL) and water (5 mL) containing potassium hydroxide (2.17 gm, 85%, 0.033 moles). Once dissolved this was diluted with water (50 mL). This was added with stirring to a solution of sodium dihydrogen phosphate (2.2 gm) and potassium nitrosodisulfonate (2.95 gm, 0.011 moles) dissolved in water (200 mL). After stirring for 30 minutes, the solution was acidified with phosphoric acid, and the mixture was extracted with methylene chloride (2×100 mL). After drying over sodium sulfate, the solution was filtered and evaporated under vacuum. The quinone was purified by chromatography on silica using 10% methanol in methylene chloride as eluent.

### Example 11

### Synthesis of Carboxymethyl Lactone

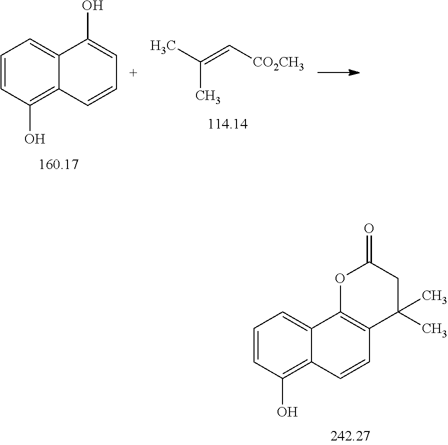

### Step 1.

A solution of 1,5-dihydroxynaphthalene (32.0 g, 0.20 moles) and methyl 3,3-dimethylacrylate (11.4 gm, 0.10 moles) in methanesulfonic acid (125 mL) was stirred and heated at 70° C. for 2 hours. After cooling, the solution was poured into ice and water (600 gm), and the precipitated oil was extracted into ethyl acetate (300 mL). The ethyl acetate solution was evaporated under vacuum, and the oily residue was stirred with methanol (100 mL) and water (100 mL). To this was added potassium hydroxide pellets (33 gm), and stirring was continued for 45 minutes. Water (300 mL) was added, and the pH was adjusted to 8.0. The mixture was extracted with ethyl acetate (2×200 mL) to remove unreacted 1,5-dihydroxynaphthalene. The aqueous was strongly acidified with phosphoric acid to a pH of about 1.0, and the mixture was heated at 70° C. for 30 minutes to promote lactonization. After cooling, the oil which had separated was extracted into t-butyl methylether (TBME, 300 mL). This solution was extracted with 5% potassium hydroxide solution (2×100 mL), and the aqueous was then acidified with 10% phosphoric acid (100 mL). The acidified aqueous was extracted with ethyl acetate (2×200 mL) and after washing with water (100 mL), the organic solution was dried (MgSO₄), filtered and evaporated under vacuum. The remaining solid was recrystallized from ethyl acetate and heptane to give the mono-lactone in a yield of 10 gm (41.3%).

### Step 2.

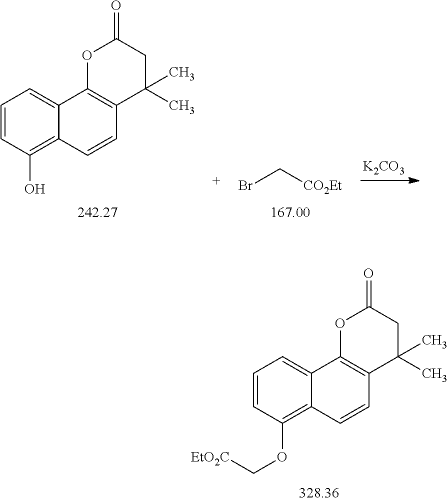

The lactone (24.2 gm, 0.10 moles), ethyl bromoacetate (16.7 gm, 0.10 moles) and potassium carbonate (20 gm) were combined in acetone (200 mL). This mixture was stirred at reflux overnight. After cooling, the acetone was removed under vacuum, and the residue was partitioned between water (200 mL) and diethyl ether (400 mL). The ether solution was washed with 5% potassium hydroxide (2×100 mL) and then with 5% phosphoric acid (100 mL). After drying over sodium sulfate, the solution was filtered and evaporated under vacuum. The remaining oil was crystallized from ethyl acetate and heptanes to give the product as a white solid in a yield of 20 gm (61%).

### Step 3.

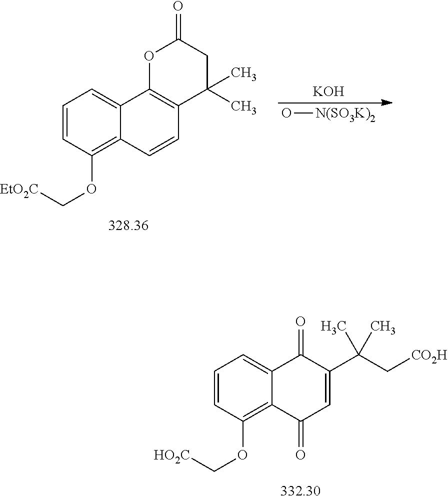

A portion of the lactone (5.09 gm, 1.55×10⁻² moles) was warmed in methanol (15 mL) and water (15 mL) containing potassium hydroxide (3.36 gm, 85%, 5.09×10⁻² moles). Once dissolved, the solution was diluted with water (75 mL).

Sodium dihydrogen phosphate dihydrate (3.42 gm) was dissolved in water (300 mL) and to this solution was added potassium nitrosodisulfonate (10.5 gm, 3.85×10⁻² moles). After stirring for 10 minutes the solution of the hydrolyzed lactone, from above, was added.

The reaction was allowed to stir for 30 minutes after which the pH was adjusted to 1.0 with phosphoric acid, and then the aqueous reaction mixture was extracted with methylene chloride (2×175 mL). The combined yellow extracts were dried (Na₂SO₄), filtered and evaporated under vacuum to give the quinone as a yellow solid in a yield of 3.2 gm (62%).

### Step 4.

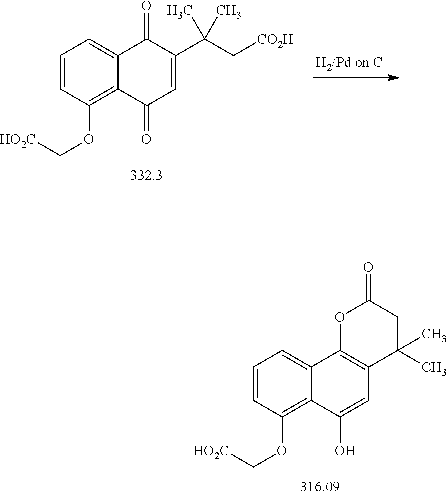

To a solution of the lactone (3.0 gm, 9.0×10⁻³ moles) in methanol (50 mL) was added 10% palladium on carbon (500 mg). This mixture was hydrogenated on a Parr apparatus at 50 psi of hydrogen. After one hour, hydrogen consumption had stopped. The mixture was filtered free of catalyst, and the filtrates were evaporated under vacuum to give the carboxymethyl lactone as a white solid in quantitative yield.

### Example 12

### Preparation of Gold Nanocarriers

### Step 1: Formation of AuNCs

An aqueous solution of 500 mL of 1 mM HAuCl₄ is heated to reflux for 10 min with vigorous stirring in a 1 L round-bottom flask equipped with a condenser. A solution of 50 mL of 40 mM of trisodium citrate is then rapidly added to the stirred solution. The resulting deep wine red solution is kept at reflux for 25-30 min. The heat is then withdrawn, and the solution is cooled to room temperature. The solution is then filtered through a 0.8 μm membrane filter to give the AuNCs in suspension. The AuNCs are characterized using visible spectroscopy and transmission electron microscopy. The AuNCs are ca. 20 nm diameter capped by citrate with peak absorption at 520 nm.

### Step 2: Direct PEG-amine Coupling to AuNCs

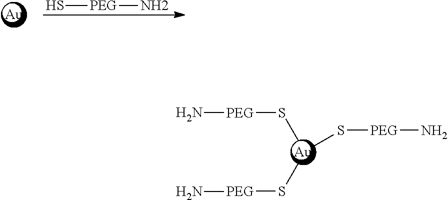

Mercaptopolyethyleneglycol amine (MW=3400) is conjugated to the AuNCs made above as follows. A solution of 145 μl of the mercaptopolyethyleneglycol amine (10 μM in 10 mM pH 9.0 carbonate buffer) is added to 1 mL of 20 nm diameter citrate-capped gold nanocarriers (1.16 nM) to produce a molar ratio of c-terminal thiol to gold of 2500:1. The mixture is stirred at room temperature under argon for 1 hour to allow complete exchange of thiol with citrate on the gold nanocarriers. The peptide-AuNC conjugates are then purified by centrifugation at 12,000 g for 30 minutes. The supernatant is decanted, and the pelleted peptide-AuNCs are washed with PBS.

### Step 3: Coupling of a Lactone to the AuNCs

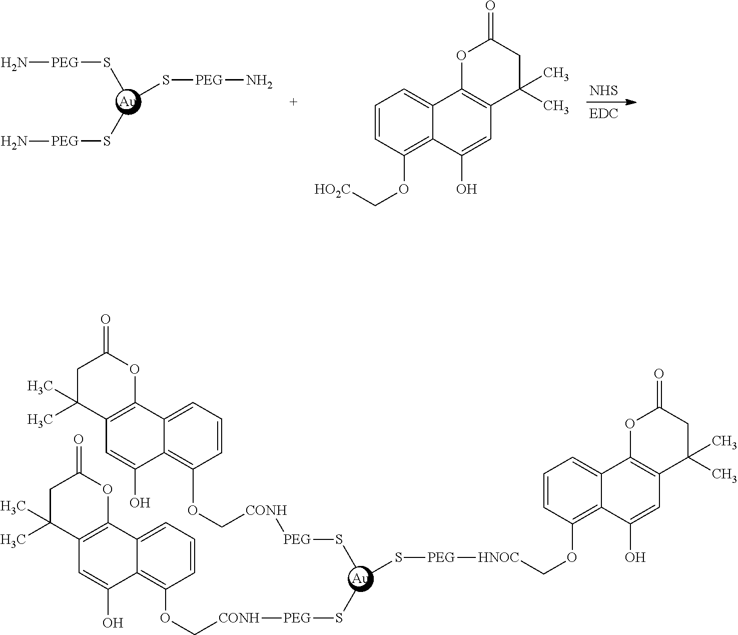

A suspension of the AuNCs from above in PBS (2 mL) is stirred as the carboxymethyl lactone (20 mg), N-hydroxysuccinimide (20 mg) and EDC (40 mg) are added. After stirring at 4° C. overnight, the derivatized AuNCs are isolated by centrifugation and are washed in PBS.

### Step 4: Generation of the Quinone AuNCs

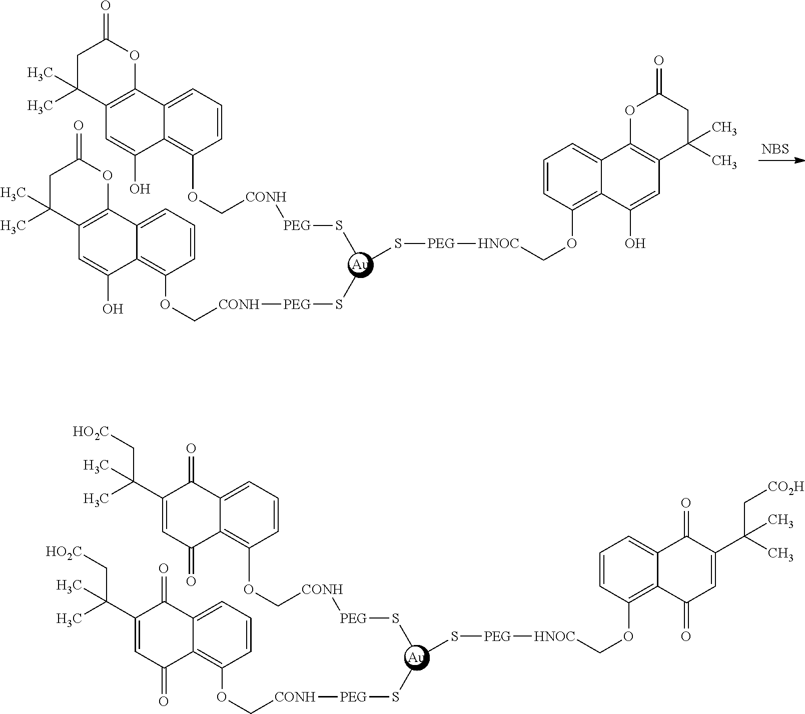

A suspension of the AuNCs from above is diluted with an equal amount of acetonitrile, and this mixture is stirred as a solution of n-bromosuccinimide (100 μL of a 10 mg/mL solution) is added. After stirring at room temperature for 1 hour, the AuNCs are isolated by centrifugation and are washed in PBS.

### Step 5: Coupling to CpG DNA

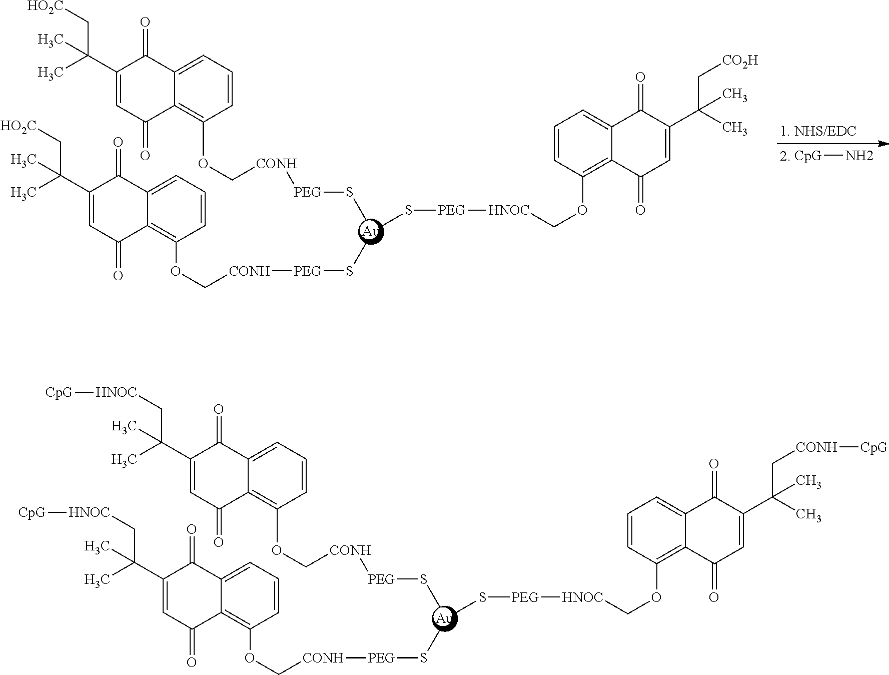

A suspension of the AuNCs from above in PBS (1 mL) is stirred as N-hydroxysuccinimide (10 mg) and EDC (20 mg) are added. After stirring at 4° C. overnight, the AuNCs are isolated by centrifugation and are washed in PBS. After re-suspending in PBS (1 mL), amino terminated CpG DNA (10 mg) is added and this mixture is stirred at 4° C. overnight. The AuNCs are isolated by centrifugation and washed in PBS.

### Example 13

### Preparation of Virus-like Particles

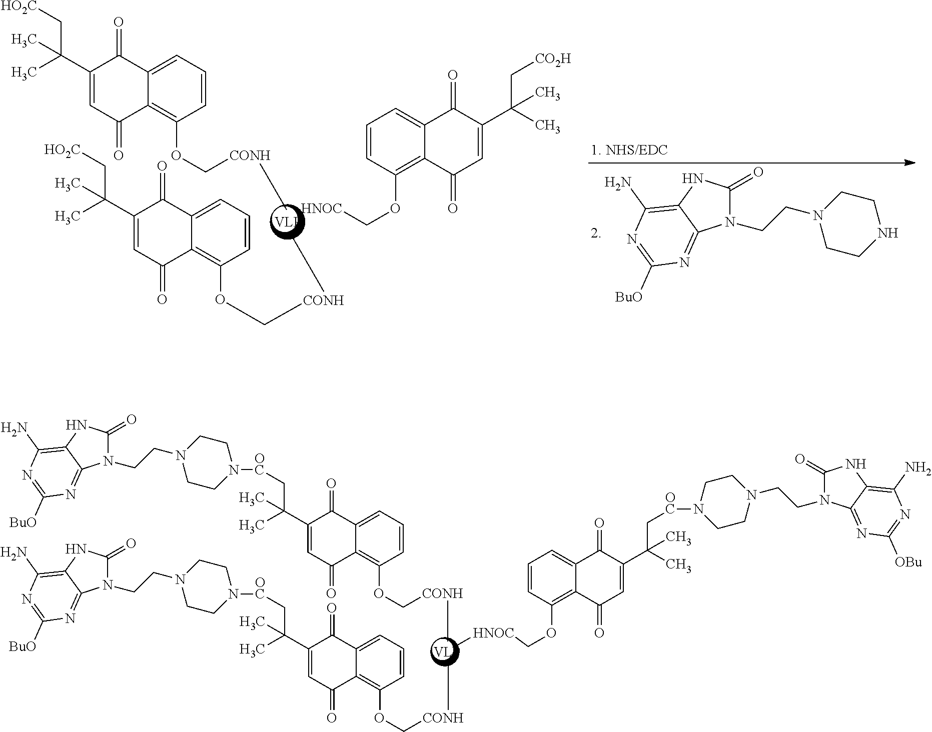

Virus-like particles (VLPs) from Cowpea mosaic virus or tobacco mosaic virus (in 20 mM HEPES, 150 mM NaCl, pH 7.2) are derivatized by incubation with a 10-fold molar excess of the carboxymethyl lactone at room temperature for 2-4 h in the presence of equimolar amounts of N-hydroxy succinimide and EDC. After removal of unreacted lactone and other byproducts by extensive dialysis against 20 mM HEPES, 150 mM NaCl (pH 7.2), the derivatized VLPs are first activated by oxidation with NBS and are then stirred for 2-4 h at 15° C. with a 5-fold molar excess of the 8-oxoadenine, NHS and EDC under argon in the dark to allow for a chemical reaction between the 8-oxoadenine piperazine nitrogen with the derivatized VLP. Uncoupled materials are then removed by extensive dialysis against PBS. The resulting VLP-8-oxoadenine conjugates are diluted with PBS for use.

### Example 14

### Preparation of PLA Conjugated to 8-Oxadenine

### Step 1.

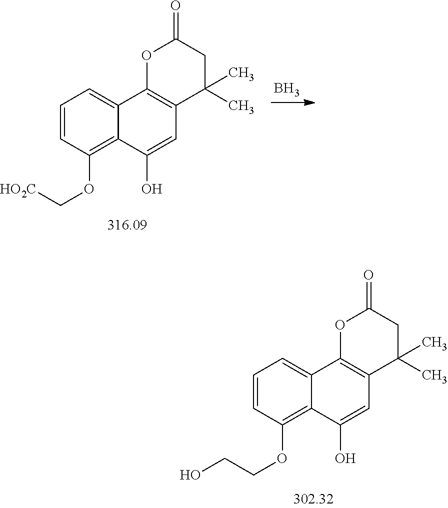

The acid (632 mg, 2×10⁻³ moles) was dissolved in dry THF (10 mL) under argon. This solution was stirred as borane in THF (4.0 mL, 1.0 M, 4×10⁻³ moles) was added via syringe in 10 minutes. The resulting solution was stirred at room temperature for 2 hours. To the reaction was added 20% hydrochloric acid (10 mL), and stirring was continued for 30 minutes. The reaction was partitioned between water (50 mL) and ethyl acetate (100 mL), and the ethyl acetate solution was washed with saturated sodium bicarbonate (50 mL) and then brine (50 mL). This solution was dried (Na₂SO₄), filtered and evaporated under vacuum. The solid residue was purified by chromatography on silica using 5% methanol in methylene chloride as eluent. 475 mg (79%) of the purified product was obtained as a white solid.

### Step 2.

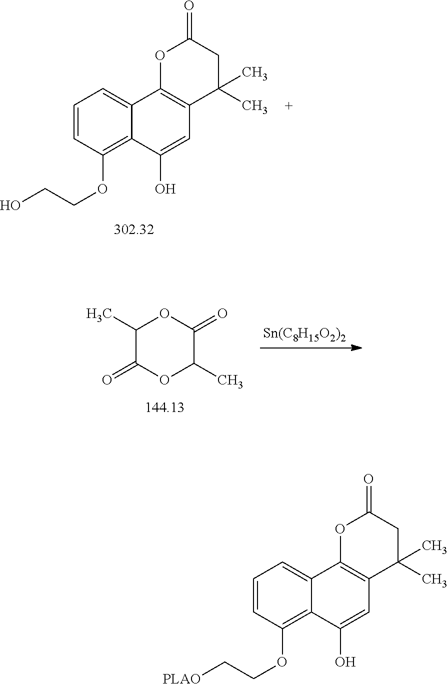

A mixture of the lactone (302 mg, 1.0×10⁻³ moles) and D/L lactide (7.0 gm, 4.86×10⁻² moles) in toluene (100 mL) was heated to reflux, and a portion of the toluene (50 mL) was distilled to dry the reaction solution. After cooling slightly under argon, tin (II) ethylhexanoate (200 μL) was added, and the solution was heated at 120° C. for 16 hours. After cooling, the toluene was evaporated under vacuum, and the remaining polymer mass was dissolved in ethyl acetate. To this solution was added a solution of ethylenediamine tetraacetic acid tetrasodium salt (10 gm) in water (10 mL). After stirring vigorously for 2 minutes, acetic acid (3.2 mL) was added, and vigorous stirring was continued. After about 15 minutes the EDTA, water and acetic acid solidified and formed precipitate in the flask. The ethyl acetate was decanted from the solid and was dried over sodium sulfate. After filtration, the ethyl acetate was removed under vacuum, and the polymer was dried under high vacuum. NMR confirmed the structure and indicated a molecular weight of about 7 KD. The yield was 6.5 gm.

### Step 3.

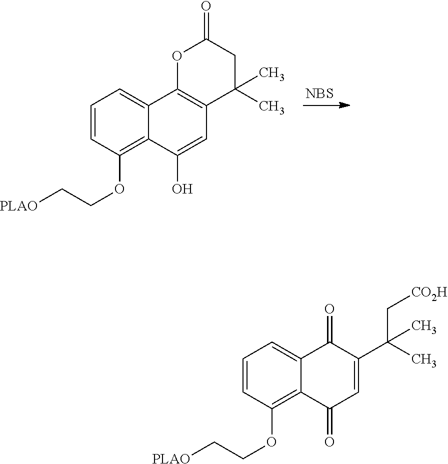

The lactone polymer (6.5 gm, 9.29×10⁻⁴ moles) was dissolved in acetonitrile (50 mL) and water (3 mL). This solution was stirred as N-bromosuccinimide (NBS, 182 mg, 1.02×10⁻³ moles) dissolved in acetonitrile (5 mL) was slowly added. Upon addition of the NBS, the colorless solution turned bright yellow. After stirring for 10 minutes, the solution was diluted with ethyl acetate (300 mL), and this solution was washed with water (2×100 mL). The ethyl acetate solution was dried over sodium sulfate, then filtered and evaporated under vacuum to a volume of 75 mL. With stirring, 2-propanol (200 mL) was slowly added which caused the polymer to precipitate as a yellow mass. The 2-propanol was decanted from the polymer and excess 2-propanol was removed under vacuum. After drying under high vacuum there was obtained 6.0 gm of polymer.

### Step 4.

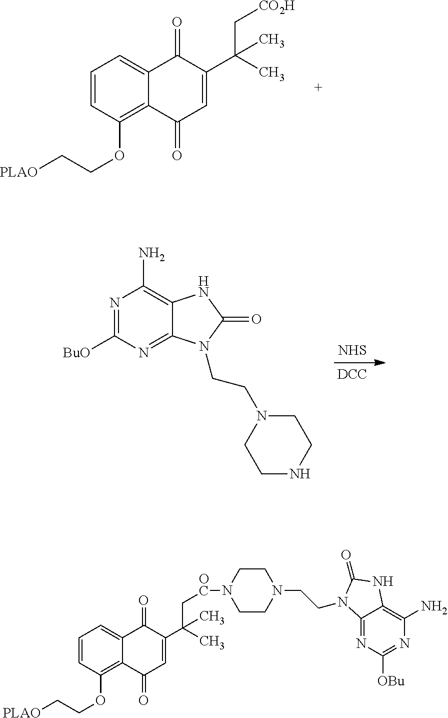

The PLA-quinone polymer (6.0 gm, 8.57×10⁻⁴ moles), N-hydroxysuccinimide (198 mg, 1.71×10⁻³ moles) and dicyclohexylcarbodiimide (354 mg, 1.71×10⁻³ moles) were dissolved in a mixture of dimethyl formamide (15 mL) and tetrahydrofuran (100 mL). The solution was stirred overnight at room temperature and was then filtered free of dicyclohexyl urea. To this solution was added the 8-oxoadenine (574 mg, 1.71×10⁻³ moles) and 4-dimethylaminopyridine (50 mg). This solution was kept at 50° C. overnight. After cooling, the reaction was evaporated under vacuum and then dissolved in ethyl acetate (300 mL). The resulting solution was washed with 5% phosphoric acid (50 mL) followed by saturated sodium bicarbonate (50 mL). After drying over magnesium sulfate, the solution was filtered and evaporated under vacuum to about 50 mL. Addition of 2-propanol (200 mL) caused the polymer to precipitate as a sticky mass. Excess 2-propanol was removed under vacuum and the residue was dried under high vacuum to provide 5 gm of the PLA-quinone-8-oxoadenine conjugate.

### Example 15

### Preparation of PLA-Quinone Conjugated Doxorubicin Anticancer Drug

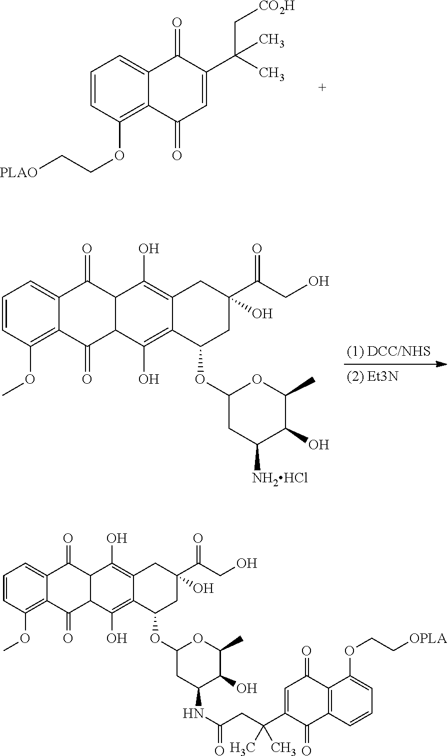

The PLA-quinone polymer from above, Step 3 (3.0 gm, 4.3×10⁻⁴ moles), N-hydroxysuccinimide (NHS) (100 mg, 0.85×10⁻³ moles) and dicyclohexylcarbodiimide (DCC) (180 mg, 0.85×10⁻³ moles) are dissolved in a mixture of dimethylformamide (DMF) (15 mL) and Tetrahydrofuran (THF) (15 mL). The solution is stirred overnight at room temperature. To this solution is then added doxorubicin hydrochloride (580 mg, 1.0×10⁻³ moles), followed by triethylamine (0.28 mL, 2×10⁻³ moles). The resulting solution is kept at room temperature overnight. The reaction is then concentrated under vacuum to remove THF. The residual is then added to 50 mL of isopropyl alcohol (IPA) to precipitate out the polymer-quinone-doxorubicin conjugate. The polymer is further washed with IPA (2×25 mL) and ether (25 mL) and dried under vacuum to give PLA-quinone-doxorubicin conjugate as an off-white solid (3.0 g)

### Example 16

### Preparation of Synthetic Nanocarriers with Covalently Coupled 8-Oxoadenine

The 8-oxoadenine is synthesized according to the synthesis provided in WO 2010/018131 A1. The PLA-quinone-8-oxoadenine conjugate is prepared as described above. PLA-PEG-nicotine conjugate is prepared as follows. PLA is prepared by a ring opening polymerization using D,L-lactide (MW=approximately 15 KD-18 KD). The PLA structure is confirmed by NMR. The polyvinyl alcohol (MW=11 KD-31 KD, 85% hydrolyzed) is purchased from VWR scientific. These were used to prepare the following solutions:

1. PLA-8-oxoadenine conjugate @ 100 mg/mL in methylene chloride
2. PLA-PEG-nicotine in methylene chloride @ 100 mg/mL
3. PLA in methylene chloride @ 100 mg/mL
4. Polyvinyl alcohol in water @50 mg/mL.

Solution #1 (0.25 to 0.75 mL), solution #2 (0.25 mL) and solution #3 (0.25 to 0.5 mL) are combined in a small vial with distilled water (0.5 mL), and the mixture is sonicated at 50% amplitude for 40 seconds using a Branson Digital Sonifier 250. To this emulsion is added solution #4 (2.0 mL), and sonication at 35% amplitude for 40 seconds using the Branson Digital Sonifier 250 forms the second emulsion. This is added to a beaker containing phosphate buffer solution (30 mL), and this mixture is stirred at room temperature for 2 hours to form the nanocarriers. To wash the nanocarriers, a portion of the nanocarrier dispersion (7.0 mL) is transferred to a centrifuge tube and spun at 5,300 g for one hour. Supernatant is removed, and the pellet is resuspended in 7.0 mL of phosphate buffered saline. The centrifuge procedure is repeated, and the pellet is resuspended in 2.2 mL of phosphate buffered saline for a final nanocarrier dispersion of about 10 mg/mL.

1. A composition, comprising:
a compound comprising the structure of formula (I):
Q-X—Y (I),
where Q comprises a synthetic nanocarrier,
X comprises a reactive moiety that is reduced in the presence of a reducing agent or reacts with a thiol, resulting in the release of Y from Q, and
Y comprises a biologically active agent.

2. The composition of claim 1, wherein the reactive moiety that is reduced in the presence of a reducing agent or reacts with a thiol comprises a disulfide linkage or quinone.

3. The composition of claim 2, wherein the reducing agent is NADH, NADPH, or a quinone reductase enzyme.

4. The composition of claim 1 wherein the compound comprises the structure of formula (II): 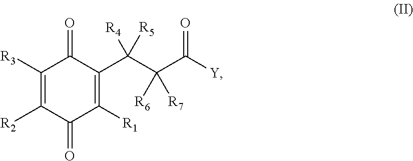
where at least one of R₁-R₇ comprises Q;
R₁ comprises Q, H, an unsubstituted or substituted alkyl, an unsubstituted or substituted aryl, alkoxy or halogen;
R₂ and R₃ each is H or comprise Q, an unsubstituted or substituted alkyl, an unsubstituted or substituted aryl, alkoxy or halogen or are joined to form a cyclic ring;
R₄ and R₅ each comprise Q, an unsubstituted or substituted alkyl, an unsubstituted or substituted aryl, alkoxy or halogen, except that both R₄ and R₅ do not comprise Q; and
R₆ and R₇ each is H or comprise Q, an unsubstituted or substituted alkyl, an unsubstituted or substituted aryl, alkoxy or halogen.

5. The composition of claim 4, wherein R₆ and R₇ each is a methyl group.

6. The composition of claim 1 wherein the compound comprises the structure of formula (III): 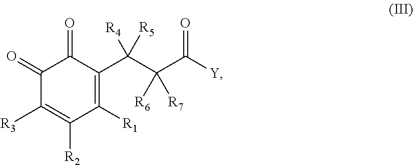
where at least one of R₁-R₇ comprises Q;
R₁ comprises Q, H, an unsubstituted or substituted alkyl, an unsubstituted or substituted aryl, alkoxy or halogen;
R₂ and R₃ each is H or comprise Q, an unsubstituted or substituted alkyl, an unsubstituted or substituted aryl, alkoxy or halogen or are joined to form a cyclic ring;
R₄ and R₅ each comprise Q or an unsubstituted or substituted alkyl, an unsubstituted or substituted aryl, alkoxy or halogen, except that both R₄ and R₅ do not comprise Q; and
R₆ and R₇ each is H or comprise Q, an unsubstituted or substituted alkyl, an unsubstituted or substituted aryl, alkoxy or halogen.

7. The composition of claim 6, wherein R₄ and R₅ each is a methyl group.

8. The composition of claim 1, wherein the reactive moiety comprises a disulfide linkage coupled to a self-immolating group.

9. The composition of claim 1, wherein Q comprises a lipid-based nanoparticle, polymeric nanoparticle, metallic nanoparticle, dendrimer, buckyball, nanowire, peptide or protein-based nanoparticle, virus-like particle or lipid-polymer nanoparticle.

10-12. (canceled)

13. The composition of claim 1, wherein Y comprises an immunomodulating agent, an anticancer agent or an antiviral agent.

14. The composition of claim 13, wherein the immunomodulating agent is a TLR agonist or CpG-containing oligonucleotide.

15. The composition of claim 14, wherein the TLR agonist comprises an imidazoquinoline or adenine compound.

16-18. (canceled)

19. The composition of claim 1, wherein Y is encapsulated within the synthetic nanocarrier, on the surface of the synthetic nanocarrier, or within and on the surface of the synthetic nanocarrier.

20-21. (canceled)

22. The composition of claim 1, further comprising a pharmaceutically acceptable excipient.

23. The composition of claim 1, further comprising another biologically active agent.

24. The composition of claim 23, wherein the other biologically active agent is at least one antigen.

25. A vaccine comprising the composition of claim 1.

26. A dosage form comprising the composition of claim 1.

27. A method comprising:
administering the composition of claim 1, or a vaccine or dosage form comprising the composition to a subject.

28. The method of claim 27, further comprising administering another biologically active agent to the subject.

29-34. (canceled)
